(19) United States
(12) Patent Application Publication
(10) Pub. No.: US 2025/0235145 A1
(43) Pub. Date: Jul. 24, 2025

(54) METHODS AND SYSTEMS TO CONFIRM DEVICE CLASSIFIED ARRHYTHMIAS UTILIZING MACHINE LEARNING MODELS

(71) Applicant: Pacesetter, Inc., Sylmar, CA (US)

(72) Inventors: Kevin J. Davis, Thousand Oaks, CA (US); Fujian Qu, San Jose, CA (US); Fady Dawoud, Studio City, CA (US)

(21) Appl. No.: 19/177,513

(22) Filed: Apr. 12, 2025

Related U.S. Application Data
(62) Division of application No. 17/341,436, filed on Jun. 8, 2021.
(60) Provisional application No. 63/094,524, filed on Oct. 21, 2020.

(51) Int. Cl. A61B 5/352; A61B 5/00; A61B 5/28; A61B 5/389

(52) U.S. Cl. CPC A61B 5/352; A61B 5/0006; A61B 5/28; A61B 5/389; A61B 5/686; A61B 5/7267; A61B 5/742

(57) ABSTRACT

System and method for declaring arrhythmias in cardiac activity are provided. The system includes memory to store specific executable instructions and a machine learning (ML) model. One or more processors are configured to execute the instructions to obtain device classified arrhythmia (DCA) data sets generated by an implantable medical device (IMD) for corresponding candidate arrhythmias episodes declared by the IMD. The DCA data sets include cardiac activity (CA) signals for one or more beats sensed by the IMD and one or more device documented (DD) markers generated by the IMD. The system applies the ML model to the DCA data sets to identify a valid sub-set of DCA data sets that correctly characterize the corresponding CA signals and an invalid sub-set of the DCA data sets that incorrectly characterize the corresponding CA signals. A display is configured to present information concerning at least one of the valid sub-set or invalid sub-set.

### RELATED APPLICATION

The present application is a divisional of U.S. application Ser. No. 17/341,436, filed 8 Jun. 2021, which claims priority to U.S. Application No. 63/094,524, filed 21 Oct. 2020, the complete subject matter of which are expressly incorporated herein by reference in their entirety.

### FIELD OF THE INVENTION

Embodiments herein relate generally to confirm device classified arrhythmias in cardiac activity signals utilizing machine learning models.

### BACKGROUND OF THE INVENTION

Today, numerous arrhythmia detection processes are implemented within implantable cardiac monitors (ICMs) that detect arrhythmias based on various criteria, such as irregularities and variation patterns in R-wave to R-wave (RR) intervals. In some embodiments, the arrhythmia detection process steps beat by beat through cardiac activity (CA) signals and analyzes the characteristics of interest, such as RR intervals over a period of time. An arrhythmia episode is declared based on the characteristics of interest, such as when the RR interval pattern for the suspect beat segments is sufficiently irregular and dissimilar from RR interval patterns for sinus beat segments. When the ICM detects an arrhythmia episode, the ICM stores the CA signals (e.g., electrocardiograms or EGM signals) associated with the episode as an arrhythmia episode (AE) data set, and includes with the AE data set one or more device documented (DD) markers designating aspects of interest within the CA signals and/or episode.

However, arrhythmia detection processes at times may declare false arrhythmia episodes when a patient is not experiencing an arrhythmia. When a false arrhythmia episode is declared, the ICM continues to store the CA signals associated with the episode as an AE data set, with the DD markers (albeit incorrect/false DD marker). False arrhythmia detection may arise due to various conditions and behavior of the heart, such as when a patient experiences sick sinus rhythms with irregular RR intervals, experiences frequent premature ventricular contractions (PVCs) and/or inappropriate R-wave sensing. In some instances, false arrhythmia detection is due, in part, to dependence upon identification of R-wave features, with little or no input concerning other features of a cardiac event. PVCs, in general, introduce unstable RR intervals, such as short-long RR intervals, where the instability may give rise to erroneous declaration of an AF episode. Thus, PVCs present a substantial challenge in connection with atrial fibrillation (AF) detection algorithms that rely on RR interval variability.

For certain implantable devices and conditions, large numbers of AE data sets may be stored and transmitted due to frequent false detections. This is particularly a challenge with implantable cardiac monitors (ICMs), in which computational power is limited and signal fidelity is often degraded. The high number of false AE places an undue burden on clinicians, who often must spend considerable time reviewing the AE data sets.

A need remains to reduce the burden placed on clinicians for reviewing EGM signals and DD markers, and in particular in connection with false arrhythmia episodes.

### SUMMARY

In accordance with embodiments herein, methods and systems train and utilize machine learning models, such as a convolutional neural network (CNN), to determine whether candidate arrhythmias (e.g., AF), declared and classified by an IMD from subcutaneous EGMs (SEGMs), are true or false positives.

In accordance with embodiments herein, a system for declaring arrhythmias in cardiac activity is provided. The system includes memory to store specific executable instructions and a machine learning (ML) model. One or more processors are configured to execute the specific executable instructions to obtain device classified arrhythmia (DCA) data sets generated by an implantable medical device (IMD) for corresponding candidate arrhythmias episodes declared by the IMD. The DCA data sets include cardiac activity (CA) signals for one or more beats sensed by the IMD and one or more device documented (DD) markers that are generated by the IMD. The system applies the ML model to the DCA data sets to identify a valid sub-set of the DCA data sets that correctly characterize the corresponding CA signals and to identify an invalid sub-set of the DCA data sets that incorrectly characterize the corresponding CA signals. The system includes a display configured to present information concerning at least one of the valid sub-set or invalid sub-set of the DCA data sets.

Optionally, the ML model may represent a convolutional neural network comprising sub-layers and including one or more 1-dimensional convolutional layer, rectified linear unit activation functions, and/or batch normalization. The CA signals may represent subcutaneous electrocardiogram (EGM) signals for a series of beats over a predetermined period of time. The one or more processors are configured to identify the one or more features of interest based in part on the CA signals aligned in time with the corresponding DD markers.

Optionally, the ML model outputs, in connection with each DCA data set, may include: i) a confidence indicator indicative of a degree of confidence that the corresponding DCA data set represents a true positive or false positive designation of an arrhythmia of interest; ii) a confidence indicator indicative of an accuracy of R-wave sensing implemented by the IMD; iii) a recommendation indicative of a sensitivity level to be utilized by the IMD to identify R waves in the CA signals; or iv) an output indicating that a particular DCA data set is unduly noisy and should not be characterized as a normal sinus rhythm, nor an arrhythmia.

Optionally, the system may include an IMD. The IMD may includes a combination of subcutaneous electrodes configured to collect the CA signals. The IMD memory may be configured to store program instructions. One or more IMD processors may be configured to execute the program instructions to: analyze the CA signals and based on the analysis declare candidate arrhythmias episodes, generate the DCA data sets including the corresponding CA signals and the corresponding DD markers and a transceiver configured to wirelessly transmit the DCA data sets to an external device.

Optionally, the system may include an external device that includes the memory and the one or more processors and a transceiver. The transceiver may be configured to wirelessly receive the DCA data sets from the IMD. The system may include a server that includes the memory and the one or more processors. The memory may be configured to store the collection of the DCA data sets. The one or more processors may apply the ML model to the collection of the DCA data sets.

In accordance with embodiments herein, a computer implemented method is provided. The method is under control of one or more processors configured with specific executable instructions. The method obtains device classified arrhythmia (DCA) data sets generated by an implantable medical device (IMD) for corresponding candidate arrhythmias episodes declared by the IMD. The DCA data sets include cardiac activity (CA) signals for one or more beats sensed by the IMD and one or more device documented (DD) markers that are generated by the IMD. The method applies a machine learning (ML) model to the DCA data sets to identify a valid sub-set of the DCA data sets that correctly characterize the corresponding CA signals and to identify an invalid sub-set of the DCA data sets that incorrectly characterize the corresponding CA signals. The method presents information concerning at least one of the valid sub-set or invalid sub-set of the DCA data sets.

Optionally, the method may apply the ML model to the CA signals from a current one of the DCA data sets. The ML model may represent a convolutional neural network comprising sub-layers and including one or more 1-dimensional convolutional layer, rectified linear unit activation functions, and/or batch normalization. The method may output a confidence indicator from the ML model in connection with each DCA data set. The confidence indicator may be indicative of a degree of confidence that the corresponding DCA data set represents a true positive or false positive designation of an arrhythmia of interest.

Optionally, the method may compare the confidence indicators for corresponding DCA data sets to a detection threshold and adding the corresponding DCA data set to the valid subset or invalid subset based on the comparison. The ML model may represent a model that is trained utilizing an augmented collection of DCA data sets. The augmented collection of the DCA data sets may include reference DCA data sets from patients and synthetic DCA data sets that are generated based on the reference DCA data sets. The ML model may represent a convolutional neural network. The method may display the CA signals and corresponding DD markers from the valid subset.

In accordance with embodiments herein, a system is provided. The system includes memory configured to store specific executable instructions. They system includes one or more processors configured to execute the specific executable instructions to: obtain reference device classified arrhythmia (DCA) data sets associated with device declared arrhythmias. The reference DCA data sets includes cardiac activity (CA) signals for one or more beats sensed by subcutaneous electrodes of an implantable medical device (IMD). The reference DCA data sets includes one or more DD markers, generated by the IMD, characterizing the CA signals within the corresponding DCA data sets. They system generates synthetic DCA data sets based on the reference DCA data sets to form an augmented collection of DCA data sets and applies the augmented collection of DCA data sets to the ML model to train the ML model.

Optionally, the one or more processors may be further configured to apply a first augmented collection of the DCA data sets that represent valid DCA data sets that include DD markers that correctly characterize the corresponding CA signals and may apply a second augmented collection of the DCA data sets that represent invalid DCA data sets that include DD markers that incorrectly characterize the corresponding CA signals. The one or more processors may be further configured to generate the synthetic DCA data sets by at least one of shifting, rotating, stretching, shrinking or applying a Gaussian component to the reference DCA data sets. The one or more processors may be further configured to generate the synthetic DCA data sets by shifting and wrapping the CA signals in the reference DCA data sets such that trailing portions of the reference DCA data sets are wrapped to form leading portions of the synthetic DCA data sets, and such that intermediate portions of the reference DCA data sets are shifted to form trailing portions of the synthetic DCA data sets.

Optionally, the one or more processors may be further configured to generate the synthetic DCA data sets by at least one of stretching or shrinking the CA signals in the reference DCA data sets by at least one of adding or subtracting an amount of time to RR intervals between successive beats in the CA signals. The synthetic DCA data sets may represent data sets that include artificially generated or computer-generated CA signals, where the CA signals and DD markers are based on the reference DCA data sets collected from a patient, but where the CA signals in the synthetic DCA data set are not collected from the patient.

In accordance with embodiments herein, a computer implemented method for building a machine learning (ML) model to confirm device documented (DD) arrhythmias is provided. The method is under control of one or more processors configured with specific executable instructions. The method obtains a collection of reference device classified arrhythmia (DCA) data sets associated with device declared arrhythmias. The reference DCA data sets includes cardiac activity (CA) signals for one or more beats sensed by subcutaneous electrodes of an implantable medical device (IMD). The reference DCA data sets includes one or more DD markers, generated by the IMD, characterizing the CA signals within the corresponding DCA data sets. The method generates synthetic DCA data sets based on the reference DCA data sets to form an augmented collection of DCA data sets and applies the augmented collection of DCA data sets to the ML model to train the ML model.

Optionally, the method may apply a first augmented collection of the DCA data sets that represent valid DCA data sets that include DD markers that correctly characterize the corresponding CA signals and may apply a second augmented collection of the DCA data sets that represent invalid DCA data sets that include DD markers that incorrectly characterize the corresponding CA signals. The method may generate the synthetic DCA data sets by shifting and wrapping the CA signals in the reference DCA data sets such that trailing portions of the reference DCA data sets are wrapped to form leading portions of the synthetic DCA data sets, and such that intermediate portions of the reference DCA data sets are shifted to form trailing portions of the synthetic DCA data sets. The method may generate the synthetic DCA data sets by at least one of stretching or shrinking the CA signals in the reference DCA data sets by at least one of adding or subtracting an amount of time to RR intervals between successive beats in the CA signals.

### BRIEF DESCRIPTION OF THE DRAWINGS

FIG. 1 illustrates an ICM intended for subcutaneous implantation at a site near the heart in accordance with embodiments herein.

FIG. 2 shows a block diagram of the ICM formed in accordance with embodiments herein.

FIG. 3 shows a high-level overview of a system formed in accordance with embodiments herein.

FIG. 4 illustrates a summary of an example ML model utilized in accordance with embodiments herein.

FIG. 5 illustrates a process for training/building a machine learning (ML) model to analyze DCA data sets, relative to an arrhythmia of interest, in accordance with embodiments herein.

FIG. 6 illustrates examples of manners in which augmentation may be applied to construct synthetic DCA data sets in accordance with embodiments herein.

FIG. 7 illustrates a process for discriminating between valid and invalid device classified arrhythmias in accordance with embodiments herein.

FIG. 8 illustrates a distributed processing system in accordance with embodiments herein.

FIG. 9 illustrates a system level diagram indicating potential devices and networks that utilize the methods and systems herein.

### DETAILED DESCRIPTION

The terms “cardiac activity signal”, “cardiac activity signals”, “CA signal” and “CA signals” (collectively “CA signals”) are used interchangeably throughout and shall mean an analog or digital electrical signal recorded by two or more electrodes positioned subcutaneous or cutaneous, where the electrical signals are indicative of cardiac electrical activity. The cardiac activity may be normal/healthy or abnormal/arrhythmic. Non-limiting examples of CA signals include ECG signals collected by cutaneous electrodes, and EGM signals collected by subcutaneous electrodes.

The term “subcutaneous” shall be below the skin surface but not within the heart and not transvenous.

The terms “device classified arrhythmia data set” and “DCA data set” are used interchangeably and shall mean a data set that includes i) CA signals collected in response to a determination by an IMD that the CA signals are indicative of an arrhythmia of interest and ii) one or more device documented markers related to one or more features of interest in the CA signals that in whole or in part were utilized by the IMD in connection with the determination of the arrhythmia of interest.

The terms “device classified normal sinus data set” and “DCNS data set” are used interchangeably and shall mean a data set that includes i) CA signals collected in response to a determination by an IMD that the CA signals are indicative of a normal sinus rhythm and ii) one or more device documented markers related to one or more features of interest in the CA signals that in whole or in part were utilized by the IMD in connection with the determination of the normal sinus rhythm.

The term “device documented marker” refers to markers that are generated by an IMD to characterize one or more features of interest within respective CA signals. Markers may be declared based on numerous criteria, such as signal processing, feature detection and arrhythmia detection software and hardware within and/or operating on the implantable cardiac monitor and/or implantable medical device.

The term “marker” shall mean data and/or information identified from CA signals that may be presented as graphical and/or numeric indicia indicative of one or more features within the CA signals and/or indicative of one or more episodes exhibited by the cardiac events. Markers may be superimposed upon CA signals or presented proximate to, and temporally aligned with, CA signals. Non-limiting examples of markers may include R-wave markers, noise markers, activity markers, interval markers, refractory markers, P-wave markers, T-wave markers, PVC markers, sinus rhythm markers, AF markers and other arrhythmia markers. As a further nonlimiting example, basic event markers may include “AF entry” to indicate a beginning of an AF event, “in AF” to indicate that AF is ongoing, “AF exit” to indicate that AF has terminated, “T” to indicate a tachycardia beat, “B” to indicate a bradycardia beat, “A” to indicate an asystole beat, “VS” to indicate a regular sinus beat, “Tachy” to indicate a tachycardia episode, “Brady” to indicate a Bradycardia episode, “Asystole” to indicate an asystole episode, “Patient activated” to indicate a patient activated episode. An activity marker may indicate activity detected by activity sensor during the CA signal. Noise markers may indicate entry/start, ongoing, recovery and exit/stop of noise. Markers may be presented as symbols, dashed lines, numeric values, thickened portions of a waveform, and the like. Markers may represent events, intervals, refractory periods, ICM activity, and other algorithm related activity. For example, interval markers, such as the R-R interval, may include a numeric value indicating the duration of the interval. The AF markers indicate atrial fibrillation rhythmic.

The term “synthetic DCA data sets” shall mean to data sets that include artificially generated or computer-generated CA signals, where the CA signals and DD markers are based on actual DCA data sets collected from a patient, but where the CA signals are not collected from an actual patient.

The terms “beat” and “cardiac event” are used interchangeably and shall include both normal or abnormal events.

The terms “normal” and “sinus” are used to refer to events, features, and characteristics of, or appropriate to, a heart's healthy or normal functioning.

The terms “abnormal,” or “arrhythmic” are used to refer to events, features, and characteristics of, or appropriate to, a un-healthy or abnormal functioning of the heart.

The term “machine learning” shall mean an artificial intelligence algorithm that learns from various automatic or manual inputs, such as features of interest, prior device classified arrhythmias, observations and/or data. The machine learning algorithm is adjusted over multiple iterations based on the features of interest, prior device classified arrhythmias, observations and/or data. For example, the machine learning algorithm is adjusted by supervised learning, unsupervised learning, and/or reinforcement learning. Non-limiting examples of machine learning algorithms are a convolutional neural network, gradient boosting random forest, decision tree, K-means, deep learning, artificial neural network, and/or the like.

The term “real-time” refers to a time frame contemporaneous with occurrence of a normal or abnormal episode. For example, a real-time process or operation would occur during or immediately after (e.g., within minutes or seconds after) a cardiac event, a series of cardiac events, an arrhythmia episode, and the like.

The term “obtain”, as used in connection with data, signals, information and the like, includes at least one of i) accessing memory of an IMD, ICM, external device or remote server where the data, signals, information, etc. are stored, ii) receiving the data, signals, information, etc. over a wireless communications link between the ICM or IMD and a local external device, iii) receiving the data, signals, information, etc. at a remote server over a network connection and/or iv) sensing signals (e.g., CA signals, impedance signals, etc.) between a combination of electrodes provide on or coupled to the ICM or IMD. An obtaining operation, when from the perspective of an ICM or IMD, may include sensing new signals in real time, and/or accessing memory to read stored data, signals, information, etc. from memory within the ICM or IMD. The obtaining operation, when from the perspective of a local external device, includes receiving the data, signals, information, etc. at a transceiver of the local external device where the data, signals, information, etc. are transmitted from an ICM and/or a remote server. The obtaining operation may be from the perspective of a remote server, such as when receiving the data, signals, information, etc. at a network interface from a local external device and/or directly from an ICM. The remote server may also obtain the data, signals, information, etc. from local memory and/or from other memory, such as within a cloud storage environment and/or from the memory of a workstation or clinician external programmer.

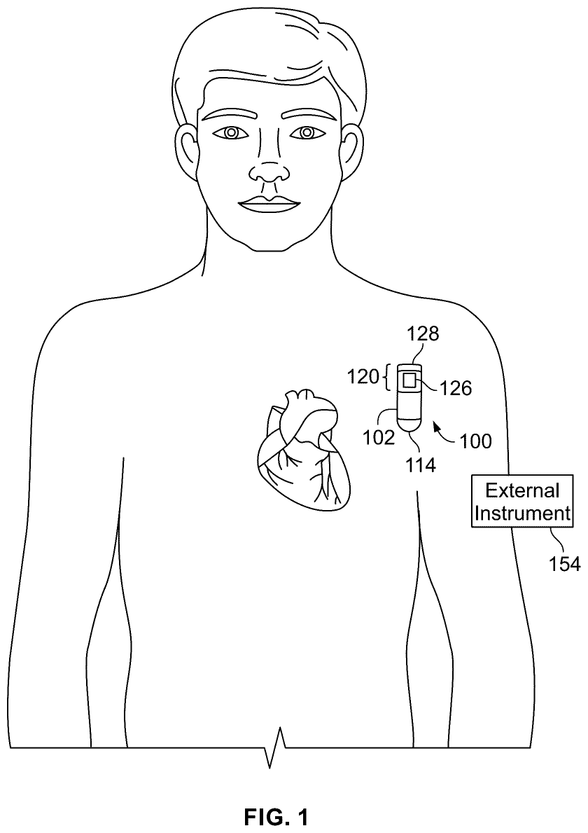

FIG. 1 illustrates an ICM 100 intended for subcutaneous implantation at a site near the heart. The ICM 100 includes a pair of spaced-apart sense electrodes 114, 126 positioned with respect to a housing 102. The sense electrodes 114, 126 provide for detection of far field electrogram signals. Numerous configurations of electrode arrangements are possible. For example, the electrode 114 may be located on a distal end of the ICM 100, while the electrode 126 is located on a proximal side of the ICM 100. Additionally or alternatively, electrodes 126 may be located on opposite sides of the ICM 100, opposite ends or elsewhere. The distal electrode 114 may be formed as part of the housing 102, for example, by coating all but a portion of the housing with a nonconductive material such that the uncoated portion forms the electrode 114. In this case, the electrode 126 may be electrically isolated from the housing 102 electrode by placing it on a component separate from the housing 102, such as the header 120. Optionally, the header 120 may be formed as an integral portion of the housing 102. The header 120 includes an antenna 128 and the electrode 126. The antenna 128 is configured to wirelessly communicate with an external device 154 in accordance with one or more predetermined wireless protocols (e.g., Bluetooth, Bluetooth low energy, Wi-Fi, etc.). The housing 102 includes various other components such as: sense electronics for receiving signals from the electrodes, a microprocessor for processing the signals in accordance with algorithms, such as the AF detection algorithm described herein, a loop memory for temporary storage of CA data, a device memory for long-term storage of CA data upon certain triggering events, such as AF detection, sensors for detecting patient activity and a battery for powering components.

In at least some embodiments, the ICM 100 is configured to be placed subcutaneously utilizing a minimally invasive approach. Subcutaneous electrodes are provided on the housing 102 to simplify the implant procedure and eliminate a need for a transvenous lead system. The sensing electrodes may be located on opposite sides of the device and designed to provide robust episode detection through consistent contact at a sensor-tissue interface. The ICM 100 may be configured to be activated by the patient or automatically activated, in connection with recording subcutaneous ECG signals.

The ICM 100 senses far field, subcutaneous CA signals, processes the CA signals to detect arrhythmias and if an arrhythmia is detected, automatically records the CA signals in memory for subsequent transmission to an external device. The CA signal processing and AF detection is provided for, at least in part, by algorithms embodied in or implemented by the microprocessor. The ICM 100 includes one or more processors and memory that stores program instructions directing the processors to implement AF detection utilizing an on-board R-R interval irregularity (ORI) process that analyzes cardiac activity signals collected over one or more sensing channels.

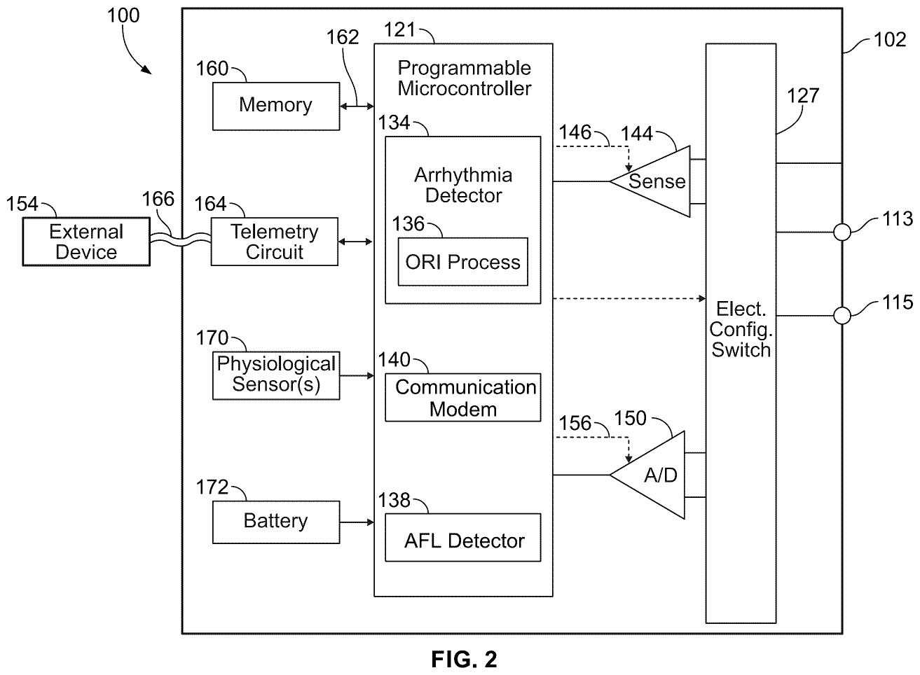

FIG. 2 shows a block diagram of the ICM 100 formed in accordance with embodiments herein. The ICM 100 may be implemented to monitor ventricular activity alone, or both ventricular and atrial activity through sensing circuit. The ICM 100 has a housing 102 to hold the electronic/computing components. The housing 102 (which is often referred to as the “can”, “case”, “encasing”, or “case electrode”) may be programmably selected to act as an electrode for certain sensing modes. Housing 102 further includes a connector (not shown) with at least one terminal 113 and optionally additional terminals 115. The terminals 113, 115 may be coupled to sensing electrodes that are provided upon or immediately adjacent the housing 102. Optionally, more than two terminals 113, 115 may be provided in order to support more than two sensing electrodes, such as for a bipolar sensing scheme that uses the housing 102 as a reference electrode. Additionally or alternatively, the terminals 113, 115 may be connected to one or more leads having one or more electrodes provided thereon, where the electrodes are located in various locations about the heart. The type and location of each electrode may vary.

The ICM 100 includes a programmable microcontroller 121 that controls various operations of the ICM 100, including cardiac monitoring. Microcontroller 121 includes a microprocessor (or equivalent control circuitry), RAM and/or ROM memory, logic and timing circuitry, state machine circuitry, and I/O circuitry. The microcontroller 121 also performs the operations described herein in connection with collecting cardiac activity data and analyzing the cardiac activity data.

A switch 127 is optionally provided to allow selection of different electrode configurations under the control of the microcontroller 121. The electrode configuration switch 127 may include multiple switches for connecting the desired electrodes to the appropriate I/O circuits, thereby facilitating electrode programmability. The switch 127 is controlled by a control signal from the microcontroller 121. Optionally, the switch 127 may be omitted and the I/O circuits directly connected to the housing electrode 114 and a second electrode 126.

Microcontroller 121 includes an arrhythmia detector 134 that is configured to analyze cardiac activity signals to identify potential arrhythmia episodes (e.g., Tachycardias, Bradycardias, Asystole, Brady pause, atrial fibrillation, etc.). By way of example, the arrhythmia detector 134 may implement an arrhythmia detection algorithm as described in U.S. Pat. No. 8,135,456, the complete subject matter of which is incorporated herein by reference. Although not shown, the microcontroller 121 may further include other dedicated circuitry and/or firmware/software components that assist in monitoring various conditions of the patient's heart and managing pacing therapies. The arrhythmia detector 134 of the microcontroller 121 includes an on-board R-R interval irregularity (ORI) process 136 that detects arrhythmia episodes, such as AF episodes using R-R interval irregularities. The ORI process 136 may be implemented as firmware, software and/or circuits. The ORI process 136 uses a hidden Markov Chains and Euclidian distance calculations of similarity to assess the transitionary behavior of one R-wave (RR) interval to another and compare the patient's RR interval transitions to the known RR interval transitions during atrial fibrillation (AF) and non-AF episodes obtained from the same patient and/or many patients.

The arrhythmia detector 134 analyzes sensed far field CA signals sensed along a sensing vector between a combination of subcutaneous electrodes for one or more beats. The arrhythmia detector 134 identifies one or more features of interest from the CA signals, and based on further analysis of the features of interest determines whether the CA signals are indicative of a normal sinus rhythm or an arrhythmia episode. When an arrhythmia episode is identified, the arrhythmia detector 134 generates one or more DD markers that are temporally aligned with corresponding features of interest in the CA signals. The arrhythmia detector 134 forms a DCA data set associated with the classified arrhythmia episode and stores the DCA data set in the memory of the IMD. The arrhythmia detector 134 iteratively or periodically repeats the analysis of incoming far field CA signals to continuously add DCA data sets for respective arrhythmia episodes, thereby forming a collection of DCA data sets.

The ICM 100 is further equipped with a communication modem (modulator/demodulator) 140 to enable wireless communication. In one implementation, the communication modem 140 uses high frequency modulation, for example using RF, Bluetooth or Bluetooth Low Energy telemetry protocols. The signals are transmitted in a high frequency range and will travel through the body tissue in fluids without stimulating the heart or being felt by the patient. The communication modem 140 may be implemented in hardware as part of the microcontroller 121, or as software/firmware instructions programmed into and executed by the microcontroller 121. Alternatively, the modem 140 may reside separately from the microcontroller as a standalone component. The modem 140 facilitates data retrieval from a remote monitoring network. The modem 140 enables timely and accurate data transfer directly from the patient to an electronic device utilized by a physician.

The ICM 100 includes sensing circuit 144 selectively coupled to one or more electrodes that perform sensing operations, through the switch 127 to detect cardiac activity data indicative of cardiac activity. The sensing circuit 144 may include dedicated sense amplifiers, multiplexed amplifiers, or shared amplifiers. It may further employ one or more low power, precision amplifiers with programmable gain and/or automatic gain control, bandpass filtering, and threshold detection circuit to selectively sense the features of interest. In one embodiment, switch 127 may be used to determine the sensing polarity of the cardiac signal by selectively closing the appropriate switches.

The output of the sensing circuit 144 is connected to the microcontroller 121 which, in turn, determines when to store the cardiac activity data of CA signals (digitized by the A/D data acquisition system 150) in the memory 160. For example, the microcontroller 121 may only store the cardiac activity data (from the A/D data acquisition system 150) in the memory 160 when a potential arrhythmia episode is detected. The sensing circuit 144 receives a control signal 146 from the microcontroller 121 for purposes of controlling the gain, threshold, polarization charge removal circuitry (not shown), and the timing of any blocking circuitry (not shown) coupled to the inputs of the sensing circuit.

Optionally, the ICM 100 may include multiple sensing circuits, similar to sensing circuit 144, where each sensing circuit is coupled to two or more electrodes and controlled by the microcontroller 121 to sense electrical activity detected at the corresponding two or more electrodes. The sensing circuit 144 may operate in a unipolar sensing configuration or in a bipolar sensing configuration. Optionally, the sensing circuit 144 may be removed entirely and the microcontroller 121 perform the operations described herein based upon the CA signals from the A/D data acquisition system 150 directly coupled to the electrodes.

The ICM 100 further includes an analog-to-digital A/D data acquisition system (DAS) 150 coupled to one or more electrodes via the switch 127 to sample cardiac activity signals across any pair of desired electrodes. The data acquisition system 150 is configured to acquire cardiac electrogram (EGM) signals as CA signals, convert the raw analog data into digital data, and store the digital data as CA data for later processing and/or telemetric transmission to an external device 154 (e.g., a programmer, local transceiver, or a diagnostic system analyzer). The data acquisition system 150 is controlled by a control signal 156 from the microcontroller 121. The EGM signals may be utilized as the cardiac activity data that is analyzed for potential arrhythmia episodes. The ACS adjustment and ORI process 136 may be applied to signals from the sensing circuit 144 and/or the DAS 150.

By way of example, the external device 154 may represent a bedside monitor installed in a patient's home and utilized to communicate with the ICM 100 while the patient is at home, in bed or asleep. The external device 154 may be a programmer used in the clinic to interrogate the ICM 100, retrieve data and program detection criteria and other features. The external device 154 may be a handheld device (e.g., smartphone, tablet device, laptop computer, smartwatch and the like) that can be coupled over a network (e.g., the Internet) to a remote monitoring service, medical network and the like. The external device 154 facilitates access by physicians to patient data as well as permitting the physician to review real-time CA signals while collected by the ICM 100.

The microcontroller 121 is coupled to a memory 160 by a suitable data/address bus 162. The programmable operating parameters used by the microcontroller 121 are stored in memory 160 and used to customize the operation of the ICM 100 to suit the needs of a particular patient. Such operating parameters define, for example, detection rate thresholds, sensitivity, automatic features, AF detection criteria, activity sensing or other physiological sensors, and electrode polarity, etc.

In addition, the memory 160 stores the cardiac activity data, as well as the markers and other data content associated with detection of arrhythmia episodes. The operating parameters of the ICM 100 may be non-invasively programmed into the memory 160 through a telemetry circuit 164 in telemetric communication via communication link 166 with the external device 154. The telemetry circuit 164 allows intracardiac electrograms and status information relating to the operation of the ICM 100 (as contained in the microcontroller 121 or memory 160) to be sent to the external device 154 through the established communication link 166. In accordance with embodiments herein, the telemetry circuit 164 conveys the DCA data sets and other information related to arrhythmia episodes to an external device.

The ICM 100 may further include magnet detection circuitry (not shown), coupled to the microcontroller 121, to detect when a magnet is placed over the unit. A magnet may be used by a clinician to perform various test functions of the housing 102 and/or to signal the microcontroller 121 that the external device 154 is in place to receive or transmit data to the microcontroller 121 through the telemetry circuits 164.

The ICM 100 can further include one or more physiologic sensors 170. Such sensors are commonly referred to (in the pacemaker arts) as “rate-responsive” or “exercise” sensors. The physiological sensor 170 may further be used to detect changes in the physiological condition of the heart, or diurnal changes in activity (e.g., detecting sleep and wake states). Signals generated by the physiological sensors 170 are passed to the microcontroller 121 for analysis and optional storage in the memory 160 in connection with the cardiac activity data, markers, episode information and the like. While shown as being included within the housing 102, the physiologic sensor(s) 170 may be external to the housing 102, yet still be implanted within or carried by the patient. Examples of physiologic sensors might include sensors that, for example, activity, temperature, sense respiration rate, pH of blood, ventricular gradient, activity, position/posture, minute ventilation (MV), and so forth.

A battery 172 provides operating power to all of the components in the ICM 100. The battery 172 is capable of operating at low current drains for long periods of time. The battery 172 also desirably has a predictable discharge characteristic so that elective replacement time can be detected. As one example, the housing 102 employs lithium/silver vanadium oxide batteries. The battery 172 may afford various periods of longevity (e.g., three years or more of device monitoring). In alternate embodiments, the battery 172 could be rechargeable. See for example, U.S. Pat. No. 7,294,108, Cardiac event micro-recorder and method for implanting same, which is hereby incorporated by reference.

The ICM 100 provides a simple to configure data storage option to enable physicians to prioritize data based on individual patient conditions, to capture significant events and reduce risk that unexpected events are missed. The ICM 100 may be programmable for pre-and post-trigger event storage. For example, the ICM 100 may be automatically activated to store 10-120 seconds of CA data prior to an event of interest and/or to store 10-120 seconds of post CA data. Optionally, the ICM 100 may afford patient triggered activation in which pre-event CA data is stored, as well as post event CA data (e.g., pre-event storage of 1-15 minutes and post-event storage of 1-15 minutes). Optionally, the ICM 100 may afford manual (patient triggered) or automatic activation for CA data. Optionally, the ICM 100 may afford additional programming options (e.g., asystole duration, bradycardia rate, tachycardia rate, tachycardia cycle count). The amount of CA data storage may vary based upon the size of the memory 160.

The ICM 100 may provide comprehensive safe diagnostic data reports including a summary of heart rate, in order to assist physicians in diagnosis and treatment of patient conditions. By way of example, reports may include episode diagnostics for auto trigger events, episode duration, episode count, episode date/time stamp and heart rate histograms. The ICM 100 may be configured to be relatively small (e.g., between 2-10 cc in volume) which may, among other things, reduce risk of infection during implant procedure, afford the use of a small incision, afford the use of a smaller subcutaneous pocket and the like. The small footprint may also reduce implant time and introduce less change in body image for patients.

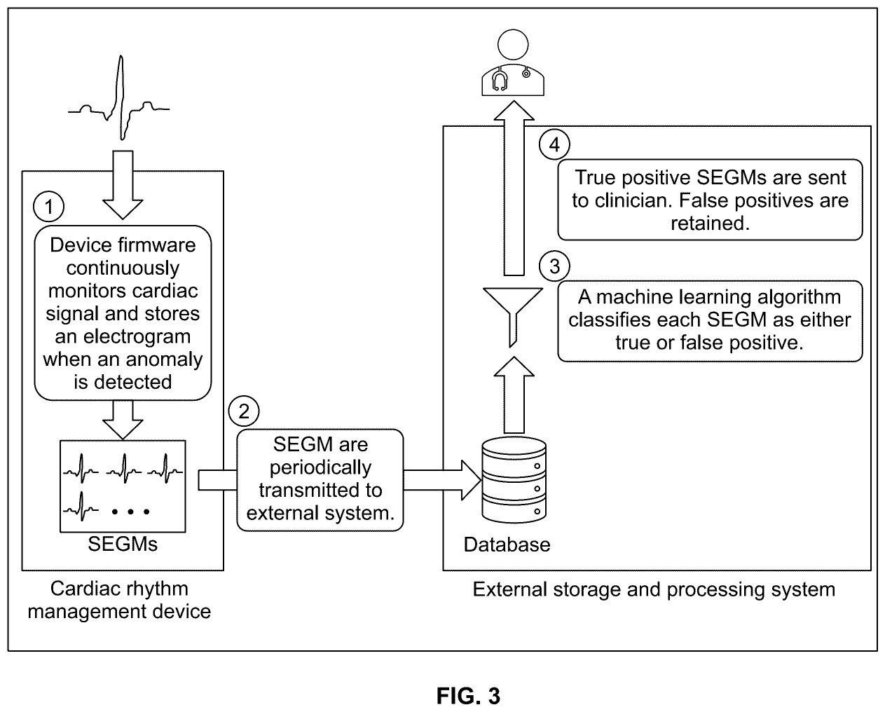

FIG. 3 shows a high-level overview of a system formed in accordance with embodiments herein. At block 1, CA signals are analyzed by one or more arrhythmia detection algorithms in the IMD. When an arrhythmia is identified, one or more DCA data sets are recorded in connection with the arrhythmia, including device documented markers designating characteristics of interest within the CA signals and/or identifying the nature of the arrhythmia. Once a collection of DCA data sets is stored in the IMD, at block 2, the collection of DCA data sets are wirelessly transmitted from the IMD to a local external device and/or a remote server. At block 3, the remote external device and/or remote server utilize one or more machine learning models to re-analyze the uploaded collection of DCA data sets. The ML model identifies valid and invalid subsets of the DCA data sets (also referred to as appropriate and inappropriate subsets). The appropriate or valid subsets include DD markers that correctly characterized the corresponding CA signals, while the inappropriate or invalid subsets includes DD markers that incorrectly characterized the corresponding CA signals. Stated another way, the appropriate or valid subset corresponds to correctly/positive arrhythmias, while the inappropriate or invalid subset corresponds to incorrect/false arrhythmias. At block 4, information concerning the true positives or valid subset is then provided to a clinician in various forms, as discussed herein.

Additionally or alternatively, the ML model may output a confidence indicator (e.g., a probability, likelihood, continuous value between 0 and 1) indicative of a level or degree of confidence that an individual underlying DCA data set represents a true positive or false positive designation of an arrhythmia. As one example, the numeric indicator may be a continuous value between 0 and 1, where the values close to zero indicate a high confidence that a subcutaneous EGM signal within the DCA data set is not indicative of an arrhythmia, and thus a false positive. The values close to 1 indicate a high confidence that a subcutaneous EGM signal within the DCA data set is indicative of an arrhythmia and thus a true positive. When a DCA data set is not indicative of an arrhythmia, the DCA data set may include CA signals that are indicative of normal sinus rhythm or otherwise. For example, the CA signals within the DCA data set may exhibit an unduly noisy signal that should not be otherwise characterized as an arrhythmia or a normal sinus rhythm.

Additionally or alternatively, the ML model may include a detection threshold that may be changed/tuned by clinicians based on the clinicians needs and various factors. In the foregoing example, where numeric values near 1 indicate a high confidence of true positives and numeric values near 0 indicate a high confidence of false positives, the detection threshold may be lowered (e.g., closer to 0) to increase the sensitivity of the ML model. For example, when the detection threshold is set at 0.25, the ML model will identify more false positive DCA data sets, as compared to when the detection threshold is set at 0.75. For example, it may be desirable to apply a higher level of sensitivity in connection with certain types of critical arrhythmias that may not occur regularly. In addition, it may be desirable to apply a higher threshold, and thus lower the sensitivity, while increasing the specificity, in connection with other types of arrhythmias that are considered less “critical” to a patient's health and that may occur more often.

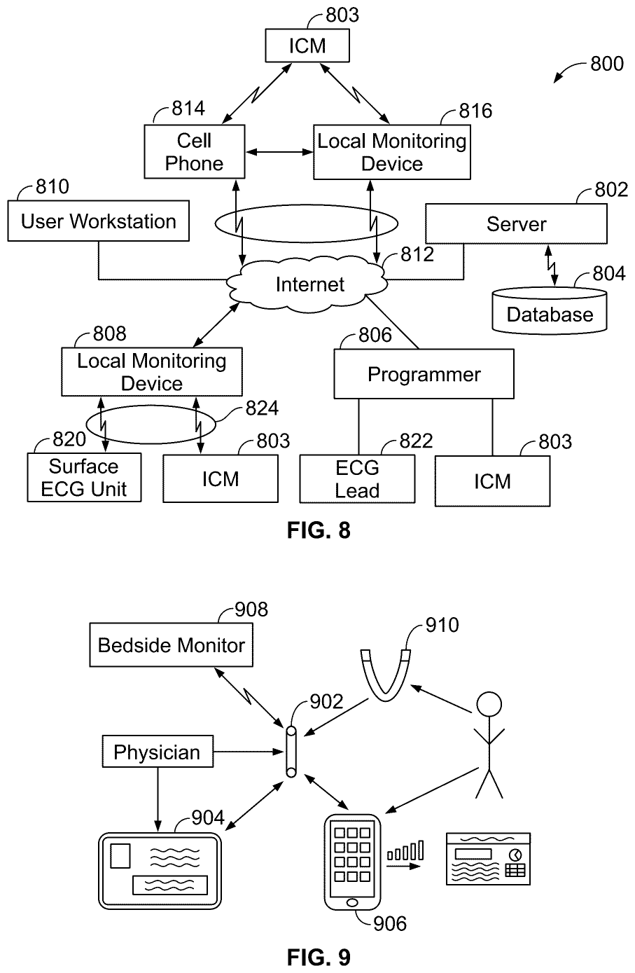

The arrhythmia detection algorithms operating on the IMD and the ML model(s) operating on the local external device and/or remote server afford two discriminators that work together to form a robust arrhythmia classification system. The system of FIG. 3 reduces false positive arrhythmias by implementing a machine learning-based confirmation process to provide a second check with respect to IMD declared arrhythmias. The process described herein may be distributed between various devices. For example, one or more of the IMD, local external device and/or remote server may include memory to store specific executable instructions and a machine learning (ML) model; and one or more processors configured to execute the specific executable instructions to: obtain device classified arrhythmia (DCA) data sets generated by an implantable medical device (IMD) for corresponding candidate arrhythmias episodes declared by the IMD, the DCA data sets including far field cardiac activity (CA) signals for one or more beats sensed by the IMD and one or more device documented (DD) markers that are generated by the IMD; and apply the ML model to the DCA data sets to identify a valid sub-set of the DCA data sets that include DD markers that correctly characterize the corresponding CA signals and to identify an invalid sub-set of the DCA data sets that include DD markers that incorrectly characterize the corresponding CA signals. One or more of the devices illustrated in FIG. 8 may further include a display configured to present information concerning at least one of the valid sub-set or invalid sub-set of the DCA data sets.

Additionally or alternatively, the one or more processors may be further configured to analyze a current one of the DCA data sets to extract one or more features of interest and to apply the one or more features of interest to the ML model. Additionally or alternatively, the one or more features of interest represent at least one of R-wave amplitude, R-wave amplitude variability, P-wave amplitude, P-wave amplitude variability, T-wave amplitudes, T-wave amplitude variability, RR interval amplitudes, RR interval amplitude variability, QRS area under the curve amplitudes, or QRS area under the curve amplitude variability, and the like. Additionally or alternatively, the CA signals represent subcutaneous electrocardiogram (EGM) signals for a series of beats over a predetermined period of time, the one or more processors configured to identify the one or more features of interest based in part on the CA signals aligned in time with the corresponding DD markers. Additionally or alternatively, the one or more devices of FIG. 3 implement an ML model that outputs, in connection with each DCA data set, at least one of: i) a confidence indicator indicative of a degree of confidence that the corresponding DCA data set represents a true positive or false positive designation of an arrhythmia of interest; ii) a confidence indicator indicative of an accuracy of R-wave sensing implemented by the IMD; iii) a recommendation indicative of a sensitivity level to be utilized by the IMD to identify R waves in the CA signals; or iv) an output indicating that a particular DCA data set is unduly noisy and should not be characterized as a normal sinus rhythm, nor an arrhythmia. One or more of the IMDs in FIG. 1 comprise a combination of subcutaneous electrodes configured to collect the CA signals; IMD memory configured to store program instructions; and one or more IMD processors configured to execute the program instructions to: analyze the CA signals and based on the analysis declare candidate arrhythmias episodes; generate the DCA data sets including the corresponding CA signals and the corresponding DD markers; and a transceiver configured to wirelessly transmit the DCA data sets to an external device. One or more of the external devices in FIG. 1 include the memory and the one or more processors and a transceiver, the transceiver configured to wirelessly receive the DCA data sets from the IMD. The server may include memory and the one or more processors, the memory configured to store the collection of the DCA data sets, the one or more processors configured to apply the ML model to the collection of the DCA data sets.

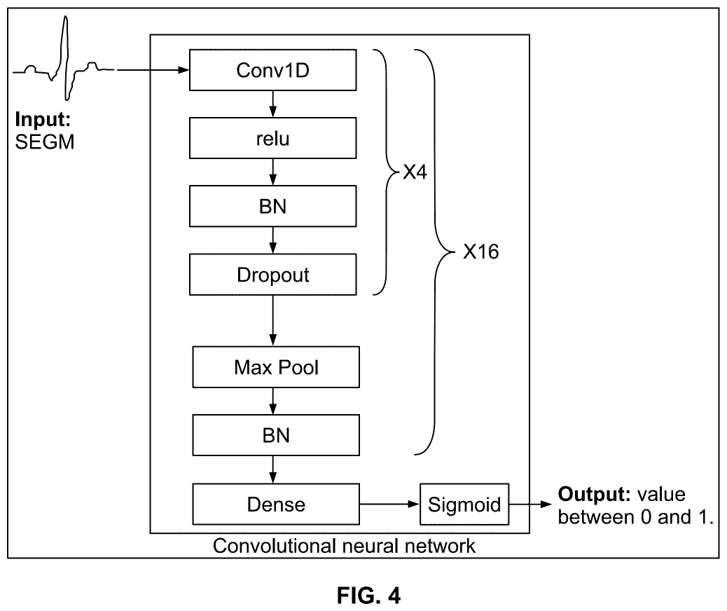

FIG. 4 illustrates a summary of an example ML model utilized in accordance with embodiments herein. The ML model includes a convolutional neural network architecture. It is recognized that the network architecture may differ and/or other types of machine learning models may be utilized. In the present form the architecture is comprised of 16 network layers, each with 4 sub-layers followed by pooling and normalization. The architecture components include: 1-dimensional convolutional layers (“Conv1D”), rectified linear unit (“relu”) activation functions, batch normalization (“BN”), dropout, etc. The network output is a continuous value between 0 and 1, where values close to zero indicate high confidence that a subcutaneous EGM signal does not correspond to an arrhythmia (e.g., is a false positive), and values close to 1 indicate high confidence of a true positive. As noted herein, the system may be tuned according to clinical needs.

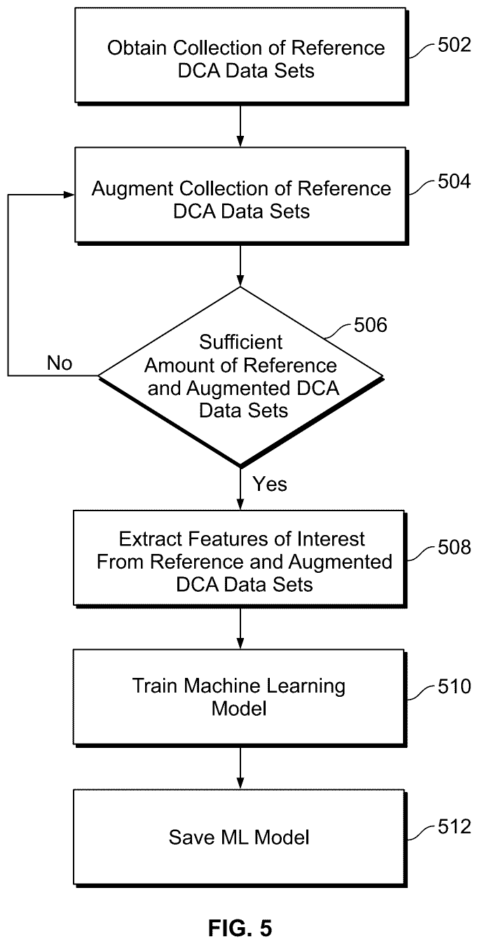

FIG. 5 illustrates a process for training/building a machine learning (ML) model to analyze DCA data sets, relative to an arrhythmia of interest, in accordance with embodiments herein. The operations of FIG. 5 may be implemented, in whole or in part by one or more processors of an IMD, local external device, remote server, and/or a combination thereof.

At 502, one or more processors of the system obtain a collection of reference DCA data sets. Each of the reference DCA data sets includes far field reference CA signals (e.g., subcutaneous EGM signals) for one or more beats sensed along a sensing vector between a combination of subcutaneous electrodes that are not located transvenously. The subcutaneous electrodes may be provided on or coupled to an IMD. For example, the subcutaneous electrodes may be provided on the housing of an ICM and/or provided on a non-transvenous lead coupled to a subcutaneous IMD. Each of the DCA data sets further includes one or more device documented (DD) markers, generated by the IMD, characterizing the CA signals within the corresponding DCA data set. A reference DCA data set include CA signals for one or more reference cardiac beats known to include a corresponding arrhythmia of interest, such as an atrial fibrillation. The reference DCA data set further include CA signals for reference cardiac beats known to be normal and to not include the arrhythmia of interest. For example, a 30 second EGM strip may be utilized as one reference DCA data set where the 30 second EGM strip is known to include an arrhythmia of interest. Multiple separate 30 second EGM strips are collected at different points in time for one patient, for a patient population, recorded by a variety of device types, device placements, device orientations and the like. where each of the separate 30 second EGM strips have CA signals that are known to include corresponding arrhythmias of interest. As a further example, a second collection of 30 second EGM strips are obtained where the second collection includes reference DCA data sets that are known to correspond to corresponding normal rhythms. The collection of reference DCA data sets may be recorded for one patient, a patient population, recorded by a variety of device types, device placements, device orientations and the like.

The reference DCA data sets include device documented markers (e.g., R-wave markers, P-wave markers, RR intervals, AF designators) that identify the cardiac beats sensed by the device within the series of cardiac events. The cardiac activity data may have been previously acquired and stored in memory of an implantable or external monitoring device, implantable or external therapy delivery device, programmer, workstation, healthcare network or other system. When the reference DCA data sets have been previously acquired, the obtaining operation at 502 represents accessing and reading the previously stored reference DCA data sets from one or more memory locations.

The CA signals are for one or more cardiac events spanning over various periods of time. As one example, multiple segments or sets of the cardiac activity data may be collected, where each segment/set is for an interval that is 30 seconds to 5 minutes in length. Optionally, the segments may include one or more IMD declared arrhythmia episodes. As another example, each of the segments or sets of the cardiac activity data may be collected for an interval that begins 10-60 seconds before an episode of interest (e.g., an AF episode) and that ends 10-60 seconds after the episode of interest. The CA signals may include one or multiple arrhythmia episodes. The DCA data sets obtained at 502 may include one or more detected arrhythmia episodes and/or one or more cardiac beats confirmed to be normal with no arrhythmia episodes. The DCA data set obtained at 502 may correspond to one continuous series of cardiac events (e.g., 1 continuous series for 30 seconds to 5 minutes) and/or separate sets of cardiac events (5, 10 or more separate series, each for 30 seconds to 5 minutes of cardiac events). Optionally, a reference DCA data set may correspond to a single beat, in which case, the CA signals correspond to one cardiac cycle, such as when the ML model is being trained to identify variability by the IMD sensing process in the detection of an R-wave.

Collection and analysis of CA signals by the IMD may be initiated automatically when the IMD detects an arrhythmia episode of interest. Additionally or alternatively, the IMD may collect and analyze CA signals in response to a user-initiated instruction or clinician. For example, a user or clinician may utilize a smart phone, programmer or other portable device to establish a communications session with the IMD and instruct the IMD to begin to collect and analyze cardiac signals, such as when the patient is experiencing discomfort, feeling faint, a rapid heart rate, during a clinic visit, etc.

At 504, the one or more processors augment the collection of reference DCA data sets, by generating synthetic DCA data sets based on the reference DCA data sets, to enlarge the total number of DCA data sets. Each of the synthetic DCA data sets is based on, but is different in some manner from, a corresponding reference DCA data set. Herein, the combination of the reference and synthetic DCA data sets are referred to as the “augmented collection of DCA data sets” and is utilized when training the ML model. By augmenting the collection of DCA data sets, embodiments herein avoid overfitting while training the ML model. The augmentation process may be performed in real time while the augmented collection of DCA data sets is supplied to the ML model.

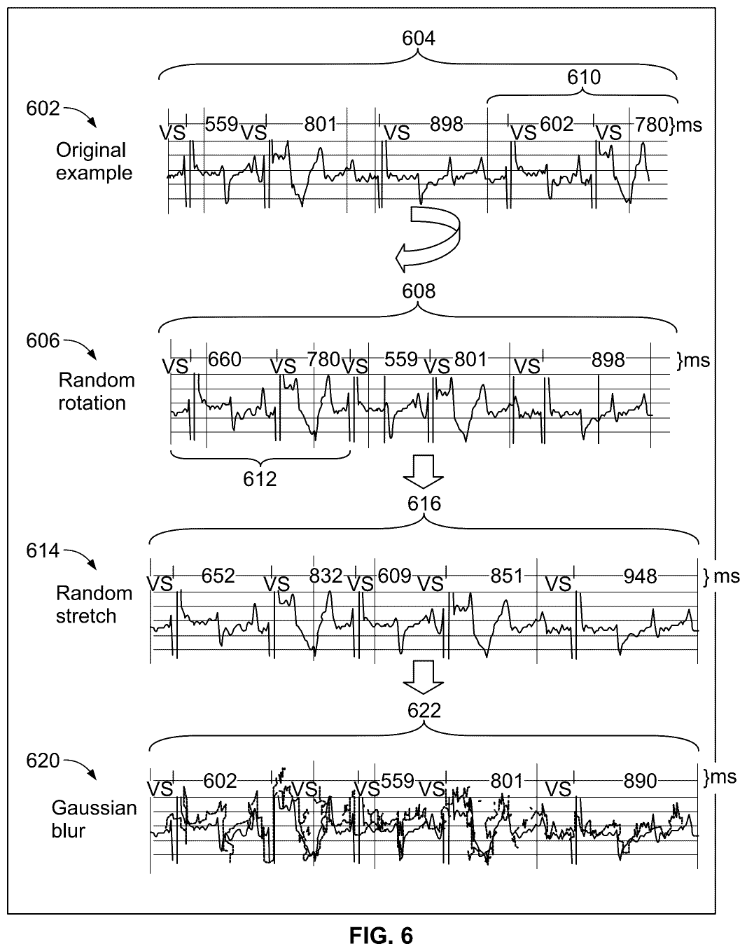

FIG. 6 illustrates examples of manners in which augmentation may be applied to construct synthetic DCA data sets in accordance with embodiments herein. In accordance with new and unique aspects herein, it has been found that a robust data augmentation strategy is an important part of real-world training of a machine learning model, such as a convolutional neural network. A convolutional neural network may be trained based on raw CA signals from the augmented collection of reference and synthetic DCA data sets. The augmentation may be implemented in various manners. As nonlimiting examples, three forms of augmentation may be used to generate synthetic DCA data sets based on the reference DCA data sets, namely random rotation, random stretch, and Gaussian blur. In FIG. 6, the top panel 602 illustrates a reference DCA data set 604 measured from a patient, where the reference DCA data set 604 includes CA signals for six heartbeats spaced apart by RR intervals of 559 ms, 801 ms, 898 ms, 602 ms and 780 ms. Device documented markers “VS” are included within the DCA data set and aligned with the peak of the R-wave for each heartbeat.

The second panel 606, in FIG. 6, illustrates a synthetic DCA data set 608 generated based on a first type of augmentation that is applied to the reference DCA data set 604, where the augmentation utilizes random rotation. The synthetic DCA data set 608 is formed by shifting and wrapping the CA signals in the reference DCA data set 604 such that a trailing portion of the reference DCA data set 604 is wrapped to form a leading portion of the synthetic DCA data set 608, and such that an intermediate portion of the reference DCA data set 604 is shifted to form a trailing portion of the synthetic DCA data set 608. In the random rotation, the one or more processors shifted and wrapped the CA signals such that a trailing portion 610 of the reference DCA data set is “wrapped” to form a leading portion 612 of the synthetic DCA data set 608. In the reference DCA data set 604, the CA signals for the last two heartbeats (in the trailing portion 610 corresponding to the RR intervals of 602 ms and 780 ms, respectively) are shifted to form the CA signals for the first two heartbeats (in the leading portion 612) in the synthetic DCA data set 608. The CA signals for the third, fourth and fifth heartbeats in the synthetic DCA data set 608, having RR intervals of 559 ms, 801 ms and 898 ms, correspond to the CA signals for the first, second and third heartbeats from the reference DCA data set 604. Optionally, the amount of rotation and/or the direction of rotation may be varied. For example, fewer or more heartbeats may be wrapped from the beginning or end of the reference DCA data set to the end or beginning of the synthetic DCA data set.

The third panel 614 illustrates another type of augmentation. In panel 614, the synthetic DCA data set 608 (generated based on random rotation) is further modified by adding a random stretch to form synthetic DCA data set 616. To form the random stretched synthetic DCA data set 616, the one or more processors space the data points defining the CA signals further apart from one another along a time axis in order to lengthen the RR interval of all or a portion of the heartbeats. In the present example, the CA signals are stretched to add a random amount of time or a predetermined amount of time (e.g., 50 ms) to the RR interval between successive beats in the CA signals. Thus, the CA signals from the random rotated synthetic DCA data set 608 are stretched to have RR intervals of 652 ms, 832 ms, 609 ms, 851 ms, and 948 ms, respectively, thereby forming the rotated/stretched synthetic DCA data set 616. Optionally, the RR intervals may be shrunken, such as by subtracting 50 ms or some other randomly determined amount of time from each RR interval. During the stretching and shrinking operation, each data point along the CA signals are correspondingly separated further from one another or compressed closer to one another along the time axis. The shrinking and/or stretching operation is applied to the entire signal, so R-R intervals are all stretched (or squeezed) to the same degree. Optionally, the stretch/shrink augmentation may be applied to the original reference DCA data set 604, and/or any other type of augmented data set.

The bottom panel 620 illustrates an example of a further augmentation applied to the random rotated, stretched synthetic DCA data set 616 through the addition of Gaussian blur. To form a Gaussian blur synthetic DCA data set 622, the one or more processors apply a Gaussian function to the data points defining the CA signals having RR intervals of 602 ms, 559 ms, 801 ms and 890 ms. For example, the Gaussian function may apply a Gaussian noise operation in one dimension, namely along the time axis. Optionally, the Gaussian noise augmentation may be applied to the original reference DCA data set 604 and/or any other type of synthetic DCA data set 608, 616. It is recognized that the foregoing types of synthetic DCA data sets are nonlimiting examples of augmentation.

Returning to FIG. 5, at 506, the one or more processors determine whether a sufficient amount of augmentation has been applied, namely whether a sufficient amount of synthetic DCA data sets have been generated. If not, flow returns to 504 where additional synthetic DCA data sets are generated. Once a desired amount of synthetic and reference DCA data sets are available, flow moves to 508. The decision at 506 enables the process to build numerous types of synthetic DCA data sets based on the reference DCA data set in order to expand the overall collection of DCA data sets by a predetermined amount. For example, it may be desirable to double, triple or otherwise enlarge the original collection of reference DCA data sets by a factor of X,

Additionally or alternatively, certain types of augmentation may be preferred over other types of augmentation. For example, it may be desirable to utilize random rotation to expand the reference DCA data sets by a first predetermined amount. If the random rotation does not achieve a sufficient amount of overall data, the process may next turn to the use of random stretches to generate additional synthetic DCA data sets. If the random rotation and random stretching does not yield a sufficient amount of overall data, the process may then perform Gaussian blurring. Additionally or alternatively, the order in which the types of augmentation may be varied. As another example, different combinations may be defined to achieve different percentages of each type of augmentation within the overall data set. For example, it may be desirable that 50% of the data is overall reference DCA data sets, 20-30% are synthetic DCA data sets generated from random rotation of the original DCA data sets, 10-15% are synthetic DCA data sets generated by random stretching of the random rotated synthetic DCA data sets, 10-15% are random stretches of the original reference DCA data set, the remainder is generated using Gaussian noise/blur, including any and all permutations and combinations thereof. At 508, an optional operation is shown that may be omitted entirely. At 508, the one or more processors analyze the augmented collection of reference and synthetic DCA data sets to identify or extract one or more features of interest from each of the data sets. As nonlimiting examples, the extracted features of interest may be features within a subcutaneous EGM signal, such as one or more of R-wave amplitude, R-wave amplitude variability, P-wave amplitude, P-wave amplitude variability, T-wave amplitudes, T-wave amplitude variability, RR interval amplitudes, RR interval amplitude variability, QRS area under the curve amplitudes, QRS area under the curve amplitude variability, and the like.

At 510, the one or more processors utilize the augmented collection of reference and synthetic DCA data sets to train a machine learning model. For example, in certain embodiments, the raw CA signals from the augmented collection of reference and synthetic DCA data sets may be applied to the machine learning model. By way of example, the machine learning model may represent a convolutional neural network that is trained as described in “Cardiologist—Level Arrhythmia Detection With Convolutional Neural Networks”, Rajpurkar, Pranav & Hannun, Awni & Haghpanahi, Masoumeh & Bourn, Codie & Ng, Andrew. (2017). ARXIV: 1707.01836v1 [cs.CV] 6 Jul. 2017, the complete subject matter of which is expressly incorporated herein by reference in its entirety.

Additionally or alternatively, the one or more processors may utilize the extracted features of interest (if obtained at 508) from the augmented collection of reference and synthetic DCA data sets to train a machine learning model.

The trained ML model may provide various types of outputs. In one example, the ML model may be trained to output simply an indication of whether a corresponding DCA data set is valid or invalid.

Additionally or alternatively, the ML model may be trained to output a confidence indicator (e.g., a probability, likelihood, continuous value between 0 and 1) indicative of a level or degree of confidence that an individual underlying DCA data set represents a true positive or false positive designation of an arrhythmia. As one example, the numeric indicator may be a continuous value between 0 and 1, where the values close to zero indicate a high confidence that a subcutaneous EGM signal within the DCA data set is not indicative of an arrhythmia, and thus a false positive. The values close to 1 indicate a high confidence that a subcutaneous EGM signal within the DCA data set is indicative of an arrhythmia and thus a true positive. When a DCA data set is not indicative of an arrhythmia, the DCA data set may include CA signals that are indicative of normal sinus rhythm or otherwise. For example, the CA signals within the DCA data set may exhibit an unduly noisy signal that should not be otherwise characterized as an arrhythmia or a normal sinus rhythm.

Additionally or alternatively, the ML model may be trained to provide an output indicating that a particular DCA data set is unduly noisy and should not be characterized as a normal sinus rhythm, nor an arrhythmia. Additionally or alternatively, the ML model may be trained to provide outputs, in connection with each DCA data set, at least one of: i) a confidence indicator indicative of a degree of confidence that the corresponding DCA data set represents a true positive or false positive designation of an arrhythmia of interest; ii) a confidence indicator indicative of an accuracy of R-wave sensing implemented by the IMD; or iii) a recommendation indicative of a sensitivity level to be utilized by the IMD to identify R waves in the CA signals.

Additionally or alternatively, the ML model may be trained to include a detection threshold that may be changed/tuned by clinicians based on the clinicians needs and various factors. In the foregoing example, where numeric values near 1 indicate a high confidence of true positives and numeric values near 0 indicate a high confidence of false positives, the detection threshold may be lowered (e.g., closer to 0) to increase the sensitivity of the ML model. For example, when the detection threshold is set at 0.25, the ML model will identify more false positive DCA data sets, as compared to when the detection threshold is set at 0.75. For example, it may be desirable to apply a higher level of sensitivity in connection with certain types of critical arrhythmias that may not occur regularly. In addition, it may be desirable to apply a higher threshold, and thus lower the sensitivity, while increasing the specificity, in connection with other types of arrhythmias that are considered less “critical” to a patient's health and that may occur more often.

At 512, the one or more processors save the machine learning model for subsequent use. The process of FIG. 5 may be implemented across a population, medical network other region to build one or more ML models. Additionally or alternatively, the machine learning models can be custom-trained per clinic. The training can be done on a regular interval or as needed. Clinics can choose to train on their own data, or on “trusted” clinics' data, or both. Performance across model versions can be compared, and clinics can optionally revert to earlier models.

For example, the ML model may represent gradient-boosting random forest that uses features of a subcutaneous EGM (SEGM) signal to train and utilize the model. The extracted features are used to construct a decision model with model confidence to perform episode classification. Additionally or alternatively, the ML model may be constructed in alternative manners and/or utilize alternative parameters. For example, the ML model may utilize an activation function such as a rectified linear (ReLu), leaky ReLu, dense, Sigmoid, tanh, etc. Network architecture could be varying number of network layers, sub-layers, hidden layers, etc. other ways of performing data augmentation. Additionally or alternatively, the ML model may be trained to perform beat-level adjudication for greater visibility into classification behavior. Additionally or alternatively, the ML model may be trained to alert clinician to inappropriate sensing and/or recommend device reprogramming.

Additionally or alternatively, the feature selection operation may be tuned/adjusted to identify an arrhythmia, such as atrial fibrillation or atrial flutter, such as by utilizing select training data sets of AF or AFL cardiac rhythms and non-AF or non-AFL rhythms.

The operations of FIG. 5 may be staged to be performed upon the CA signals at various times, such as in real time (e.g., during or shortly after a patient experiences an episode) or at any time after storage of the CA signals. The operations of FIG. 5 may be performed by devices and systems at various proximity to a patient with the ICM. For example, the CA signals may be read out of an ICM and transmitted to a local portable external device (e.g., smartphone, table computer, laptop computer, smartwatch, etc.), where the local portable external device locally implements all or a portion of the operations described in connection with FIG. 5 while in close proximity to the patient.

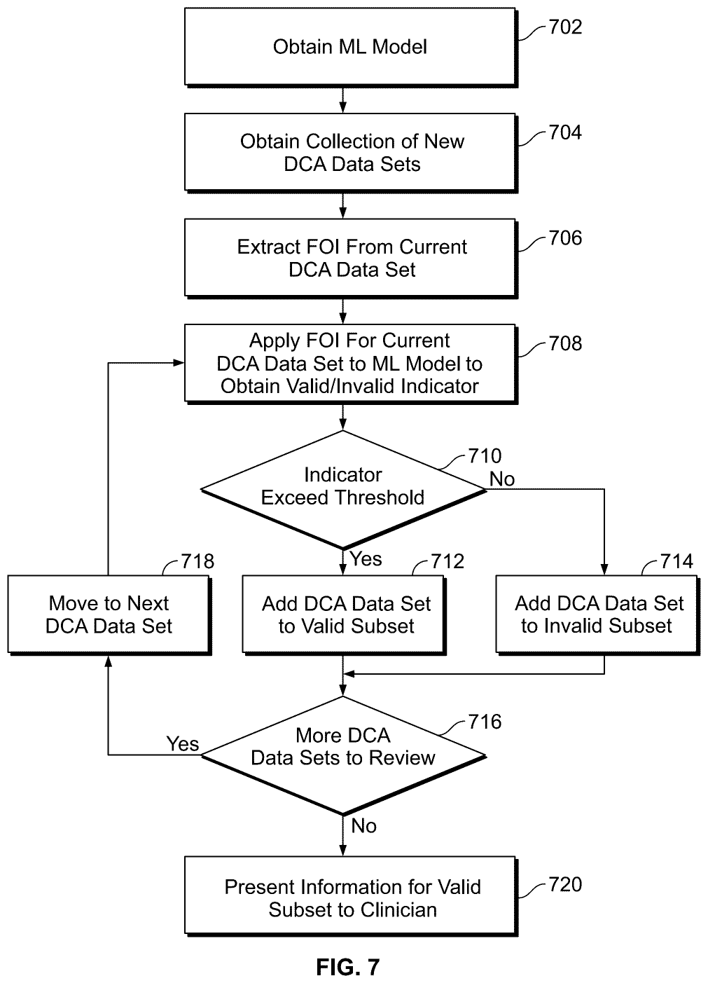

FIG. 7 illustrates a process for discriminating between valid and invalid device classified arrhythmias in accordance with embodiments herein. The operations of FIG. 7 may be implemented, in whole or in part by one or more processors of an IMD, local external device, remote server, and/or a combination thereof.

At 702, the one or more processors of the system obtain one or more ML models to be utilized in connection with analyzing previously acquired DCA data sets. For example, an external device may obtain an ML model from a collection of ML models, saved in the memory of the external device and/or at a remote server. The ML model selected from the collection may be chosen based on the type of arrhythmia in the DCA data sets (e.g., one ML model for AF, and a different ML model for AFL). Additionally or alternatively, the ML model may be selected based on the type or model of IMD, type or version of the arrhythmia detection algorithm utilized by the IMD and the like. Additionally or alternatively, the ML model may be selected based upon the clinician or clinic that will be reviewing the DCA data sets, based upon characteristics of the patient (e.g age, sex, weight, ethnic background, medical history, types of implanted devices, duration of device implant).

At 704, one or more processors of the system obtaining a collection of DCA data sets generated by an IMD for a corresponding collection of candidate arrhythmia episodes declared by the IMD. The DCA data sets may also be referred to as “candidate” DCA data sets as the machine learning model has not yet verified the determination by the IMD that an arrhythmia was in fact occurring. Each DCA data set may correspond to a separate candidate arrhythmia episode. Additionally or alternatively, a subset of the DCA data sets may correspond to related candidate arrhythmia episodes, such as when a patient is experiencing storm type arrhythmia episodes in which multiple related arrhythmia episodes occur successively. The DCA data sets may be generated by the IMD over a period of time prior to implementation of the operations of FIG. 7, and/or generated in real time by the IMD during the operations of FIG. 7. For example, an IMD may monitor a patient over several hours, days, weeks or longer and collect DCA data sets. Periodically, the previously acquired DCA data sets may be wirelessly transmitted (uploaded) from the IMD to an external device for storage and/or further transmission to a remote server. The obtaining operation at 704 may correspond simply to an external device and/or remote server accessing a previously stored collection of DCA data sets. Additionally or alternatively, the obtaining operation at 704 may at least partially include wireless reception of the DCA data sets at the external device and/or receipt of the DCA data sets at a remote server (e.g., over a network).

Each of the DCA data sets includes far field cardiac activity (CA) signals for one or more beats sensed by an implantable medical device (IMD) and one or more device documented (DD) markers that are generated by the IMD. For example, a single DCA data set corresponds to one arrhythmia episode and/or a series of arrhythmia episodes occurring successively in a short period of time. For example, when the arrhythmia of interest represents atrial fibrillation and/or atrial flutter, one DCA data set may correspond to a single AF or AFL episode, where the episode and corresponding DCA data set extend over a series of heartbeats (e.g., 7-10 beats, or until the episode has terminated). Additionally or alternatively, the DCA data set may be defined to include CA signals over a predetermined period of time following onset of the arrhythmia episode. For example, when an AF or AFL episode is detected by an AFL detector 138, the IMD may store a 30 second SEGM strip of CA signals in the single DCA data set.

At 706, the one or more processors analyze a current one of the DCA data sets to extract one or more features of interest. As noted herein, the extracted features of interest may be features within a subcutaneous EGM signal. The one or more features of interest may represent at least one of R-wave amplitude, R-wave amplitude variability, P-wave amplitude, P-wave amplitude variability, T-wave amplitudes, T-wave amplitude variability, RR interval amplitudes, RR interval amplitude variability, QRS area under the curve amplitudes, QRS area under the curve amplitude variability, and the like. For example, a single DCA data set may include CA signals for a series of beats over 30 seconds. At 704, the one or more processors identify the peak amplitude of each R-wave that was previously identified by the IMD and marked with a DD marker (e.g., “VS”). The peak amplitudes of the R waves may represent the feature of interest to be delivered to the ML model.

The DD markers are temporally aligned in time with the corresponding features within the CA signals. Optionally, the one or more processors may identify the one or more features of interest based in part on the CA signals aligned in time with the corresponding DD markers. For example, when the amplitude and/or amplitude variability in the R-wave, P-wave and/or T-wave are to be utilized as features of interest, the one or more processors may utilize positions of an R-wave marker, P-wave marker and/or T-wave marker to identify the peak amplitudes and variability.

Additionally or alternatively, the one or more processors may further compare the peak amplitudes of the R waves to identify variability between the R waves. The variability between the R waves may represent the feature of interest to be delivered to the ML model and/or an additional feature of interest to be delivered to the ML model in combination with the R-wave peak amplitudes. Additionally or alternatively, the one or more processors may identify P-wave amplitude peaks and/or T-wave amplitude peaks corresponding to DD markers identified by the IMD. The P-wave and/or T-wave amplitude peaks (alone or in combination with P-wave and T-wave amplitude peak variability) may be delivered to the ML model as alternative or additional features of interest. Additionally or alternatively, the one or more processors may identify, as a feature of interest, a DD marker designating the nature or type of arrhythmia that was previously identified by the IMD for the corresponding CA signals. For example, the IMD may designate a 30 second strip of SEGM signals to be indicative of AF. The AF DD marker may be provided as a feature of interest to the ML model.

At 708, the one or more processors apply the one or more features of interest for the current DCA data set to the ML model and in response thereto obtain a confidence indicator, from the ML model, indicative of a level or degree of confidence that the current DCA data set represents a true or false positive designation of the arrhythmia. For example, the confidence indicator may represent a probability or likelihood that the corresponding DCA data set corresponds to the arrhythmia or corresponds to a normal sinus rhythm. Additionally or alternatively, the confidence indicator may represent a numeric indicator between 0 and 1, where the values close to zero indicate a high confidence that a subcutaneous EGM signal within the DCA data set is not indicative of an arrhythmia (e.g., a normal sinus rhythm, an unduly noisy signal that should not be otherwise characterized), and thus a false positive. The values close to 1 indicate a high confidence that a subcutaneous EGM signal within the DCA data set is indicative of an arrhythmia and thus a true positive. The ML model may output, in connection with each DCA data set, at least one of: i) a confidence indicator indicative of a degree of confidence that the corresponding DCA data set represents a true positive or false positive designation of an arrhythmia of interest; ii) a confidence indicator indicative of an accuracy of R-wave sensing implemented by the IMD; iii) a recommendation indicative of a sensitivity level to be utilized by the IMD to identify R waves in the CA signals; or iv) an output indicating that a particular DCA data set is unduly noisy and should not be characterized as a normal sinus rhythm, nor an arrhythmia.

At 710, the one or more processors compare the confidence indicator provided by the ML model to a detection threshold. In the foregoing example, the confidence indicator may correspond to numeric values near 1 indicate a high confidence of true positives, to numeric values near 0 indicate a high confidence of false positives, and any value there between. The detection threshold that may be changed/tuned by clinicians based on the clinicians needs and various factors. The detection threshold may be lowered (e.g., closer to 0) to increase the sensitivity of the ML model. For example, when the detection threshold is set at 0.25, the ML model will identify a larger number of false positive DCA data sets, as compared to when the detection threshold is set at 0.75. For example, it may be desirable to apply a higher level of sensitivity in connection with certain types of critical arrhythmias that may not occur regularly. In addition, it may be desirable to apply a higher threshold, and thus lower the sensitivity, while increasing the specificity, in connection with other types of arrhythmias that are considered less “critical” to a patient's health and that may occur more often. When the confidence indicator exceeds the threshold, flow moves to 712. When the confidence indicator does not exceed the threshold, flow moves to 714.

At 712, the one or more processors add the DCA data set to a list of valid data sets to begin building a valid subset of DCA data sets. At 714, the one or more processors add the DCA data set to a list of invalid data sets to begin building and in valid subset of DCA data sets.

At 716, the one or more processors determine whether additional DCA data sets existed to be examined from the collection. If so, flow moves to 718. Otherwise, flow moves to 720. At 718, the one or more processors move to the next DCA data set within the collection and the operations at 708-716 are repeated.

The operations of FIG. 7 are iteratively repeated until all of the DCA data sets have been analyzed by the ML model and grouped into the valid or invalid subset.

At 720, the one or more processors then present information to a clinician related to at least the valid subset of DCA data sets. The presented information may vary. For example, the information may merely representative displaying and allowing the clinicians to analyze each of the DCA data sets and the valid subset. Additionally or alternatively, the information may include content related to the invalid subset. For example, the clinician may be informed that 80% of the DCA data sets were identified by the ML model to be valid, while 20% were identified to be invalid. The information may include an option to allow the clinician to view the invalid subset of DCA data sets.

Additionally or alternatively, the information may include observations and/or recommendations regarding the threshold utilized in the process of FIG. 7 to allow the clinician to adjust the level of sensitivity and/or specificity. For example, the clinician may be presented with the option to adjust the threshold. In response thereto, the process of FIG. 7 may repeat at least the decision and subset classification at operations 710-716 to “reclassified” a portion of the invalid subset. For example, when the clinician is informed that 50% of the DCA data sets were initially identified to be invalid, the clinician may choose to adjust the threshold (e.g., changing from 0.6 to 0.75). With the adjusted threshold, the process may reclassify the invalid subset, for example thereafter informing the clinician that now only 20% of the DCA data sets are determined to be invalid. Additionally or alternatively, a role numeric count of the number of DCA data sets may be provided instead of a percentage (e.g., the valid list includes 75 DCA data sets and the invalid list includes 13).

Additionally or alternatively, the information may include observations or recommendations regarding potential adjustments that may be made to the parameters of the IMD to reduce the number of false positives. For example, the ML model may output an indication that the IMD may be performing inappropriate sensing, such as over sensing or under sensing P waves, R waves, T waves and the like. As a further indication, the ML model may identify an accuracy of the R-wave sensing implemented by the IMD. Additionally or alternatively, the ML model may output recommendations for how to reprogram parameters of the IMD, such as how to reprogram sensitivity threshold utilized in connection with identifying P waves, R waves and T waves.

In the foregoing examples, an individual DCA data set is described in connection with a series of beats, and thus an individual DCA data set includes CA signals for two or more beats, with the corresponding features of interest. Additionally or alternatively, an individual DCA data set may correspond to a single beat, with the features of interest corresponding to features within a single beat (e.g., R-wave, P-wave and/or T-wave peak, duration, area under the curve). The ML model may be utilized to perform beat level adjudication, wherein the ML model analyzes each individual DCA data set. For example, the ML model may determine whether the peak(s) of the R-wave, P-wave and/or T-wave were correctly identified and labeled with DD markers, thereby affording greater visibility into the classification decisions made by the IMD.

Optionally, the operations of FIGS. 5 and 7 may be repeated for different architectures of machine learning models. For example, when the machine learning model represents a convolutional neural network, various aspects of the CNN may be varied. For example, a first CNN msy utilize a relu activation function, while a second CNN utilizes a leaky relu activation function and a third CNN utilizes a sigmoidal activation function. Additionally or alternatively, a first CNN may utilize a first number of network layers, sublayers, hidden layers and the like, while a second CNN may utilize a second number of network layers, sublayers, hidden layers and the like.

The operations of FIGS. 5 and 7 may be performed at regular intervals or as needed. Clinicians may choose to utilize their own DCA and DCNS data sets to train ML models (which are augmented as described herein), such that the training data may be specific to a clinician, a single clinic, medical facility, medical network or otherwise. Additionally or alternatively, clinicians may choose to utilize a larger patient population of DCA and DCNS data sets to train the ML models, such as by utilizing data sets collected by “trusted” third parties, or a combination thereof.

Optionally, the performance between ML models trained based on different augmented DCA data sets and/or different types/versions of models may be compared. For example, a clinician may apply the same augmented DCA data sets to a new version/type of ML model that were previously used with the prior ML model. The clinician may then be allowed to choose whether to use a newer version/types of mL model based on whether the performance is better than the prior model.

While the foregoing examples related to FIG. 7 are described in connection with analyzing DCA data sets related to arrhythmias designated by an IMD, it is recognized that the operations of FIG. 7 may be further applied to DCNS data sets collected by the IMD in connection with cardiac beats designated by the IMD to exhibit normal sinus rhythm. For example, one or more DCNS data sets may be analyzed separately from or in combination with the DCA data sets.

In the foregoing examples, the processors and memory are described to be housed within an implantable device, such as an ICM and/or IMD. Additionally or alternatively, the processors and memory may be housed within at least one of a local external device and a remote server.

FIG. 8 illustrates a distributed processing system 800 in accordance with embodiments herein. The distributed processing system 800 includes a server 802 connected to a database 804, a programmer 806, a local monitoring device 808 (e.g., IMD 100) and a user workstation 810 electrically connected to a network 812. Any of the processor-based components in FIG. 6 (e.g., workstation 810, cell phone 814, local monitoring device 816, server 802, programmer 806) may perform the processes discussed herein.

The network 812 may provide cloud-based services over the internet, a voice over IP (VoIP) gateway, a local plain old telephone service (POTS), a public switched telephone network (PSTN), a cellular phone-based network, and the like. Alternatively, the communication system may be a local area network (LAN), a medical campus area network (CAN), a metropolitan area network (MAN), or a wide area network (WAM). The communication system serves to provide a network that facilitates the transfer/receipt of data and other information between local and remote devices (relative to a patient). The server 802 is a computer system that provides services to the other computing devices on the network 812. The server 802 controls the communication of information such as DCA data sets, CA signals, motion data, bradycardia episode information, asystole episode information, arrythmia episode information, markers, heart rates, and device settings. The server 802 interfaces with the network 812 to transfer information between the programmer 806, local monitoring devices 808, 816, user workstation 810, cell phone 814 and database 804. For example, the server 802 may receive DCA data sets from various clinics, medical networks, individual patient's and the like and utilize the DCA data sets to train new ML models, update existing versions of ML models and add further outputs to existing and new ML models. The server 802 may further push new ML models and/or updated versions of ML models to various other devices, such as the programmers, local monitoring devices, cell phones, workstations and the like illustrated in FIG. 8. The database 804 stores information such as DCA data sets, arrythmia episode information, arrythmia statistics, diagnostics, DD markers, CA signal, heart rates, device settings, and the like, for a patient population, as well as separated for individual patients, individual physicians, individual clinics, individual medical networks and the like. The server 802 may implement the machine learning training operations described in connection with FIG. 5 and/or utilize the machine learning models to analyze the subsequent DCA data sets as described in connection with FIG. 7. The database 804 also maintains the machine learning models trained and updated as described herein. The machine learning models and other information are downloaded into the database 804 via the server 802 or, alternatively, the information is uploaded to the server 802 from the database 804. The programmer 806 may reside in a patient's home, a hospital, or a physician's office. The programmer 806 may wirelessly communicate with the IMD 803 and utilize protocols, such as Bluetooth, GSM, infrared wireless LANs, HIPERLAN, 3G, satellite, as well as circuit and packet data protocols, and the like. Alternatively, a telemetry “wand” connection may be used to connect the programmer 806 to the IMD 803. The programmer 806 is able to acquire ECG 822 from surface electrodes on a person (e.g., ECGs), electrograms (e.g., EGM) signals from the IMD 803, and/or CA data, arrythmia episode information, arrythmia statistics, diagnostics, markers, CA signal waveforms, atrial heart rates, device settings from the IMD 803. The programmer 806 interfaces with the network 812, either via the internet, to upload the information acquired from the surface ECG unit 820, or the IMD 803 to the server 802.

The local monitoring device 808 interfaces with the communication system to upload to the server 802 one or more of the DCA data sets, CA signals, motion data, arrythmia episode information, arrythmia statistics, diagnostics, markers, heart rates, sensitivity profile parameter settings and detection thresholds. In one embodiment, the surface ECG unit 820 and the IMD 803 have a bi-directional connection 824 with the local RF monitoring device 808 via a wireless connection. The local monitoring device 808 is able to acquire surface ECG signals from an ECG lead 822, as well as DCA CA data sets and other information from the IMD 803. On the other hand, the local monitoring device 808 may download the data and information discussed herein from the database 804 to the IMD 803.

The user workstation 810, cell phone 814 and/or programmer 806 may be utilized by a physician or medical personnel to interface with the network 812 to download DCA data sets, CA signals, motion data, and other information discussed herein from the database 804, from the local monitoring devices 808, 816, from the IMD 803 or otherwise. Once downloaded, the user workstation 810 may process the DCA data sets, CA signals and motion data in accordance with one or more of the operations described above. The user workstation 810, cell phone 814 and/or programmer 806, may be used to present information concerning at least one of the valid subset are invalid subset of the DCA data sets to a physician. Additionally or alternatively, the user workstation 810, cell phone 814 and/or programmer 806 may be utilized to display, in connection with each DCA data set, at least one of: i) a confidence indicator indicative of a degree of confidence that the corresponding DCA data set represents a true positive or false positive designation of an arrhythmia of interest; ii) a confidence indicator indicative of an accuracy of R-wave sensing implemented by the IMD; iii) a recommendation indicative of a sensitivity level to be utilized by the IMD to identify R waves in the CA signals; or iv) an output indicating that a particular DCA data set is unduly noisy and should not be characterized as a normal sinus rhythm, nor an arrhythmia.

The user workstation 810, cell phone 814 and/or programmer 806 may upload/push settings (e.g., sensitivity profile parameter settings), IMD instructions, other information and notifications to the cell phone 814, local monitoring devices 808, 816, programmer 806, server 802 and/or IMD 803. For example, the user workstation 810 may provide instructions to the IMD 803 in order to update sensitivity profile parameter settings when the IMD 803 determines that the motion data is indicative of at least one of a posture change or a respiration cycle that reduced the amplitude of the CA signals by an amount sufficient to cause the COI to exceed the COI limit.

The processes described herein in connection with reducing false positive arrhythmias may be performed by one or more of the devices illustrated in FIG. 8, including but not limited to the IMD 803, programmer 806, local monitoring devices 808, 816, user workstation 810, cell phone 814, and server 802. The process described herein may be distributed between the devices of FIG. 8. For example, one or more of the devices illustrated in FIG. 8 may include memory to store specific executable instructions and a machine learning (ML) model; and one or more processors configured to execute the specific executable instructions to: obtain device classified arrhythmia (DCA) data sets generated by an implantable medical device (IMD) for corresponding candidate arrhythmias episodes declared by the IMD, the DCA data sets including far field cardiac activity (CA) signals for one or more beats sensed by the IMD and one or more device documented (DD) markers that are generated by the IMD; and apply the ML model to the DCA data sets to identify a valid sub-set of the DCA data sets that include DD markers that correctly characterize the corresponding CA signals and to identify an invalid sub-set of the DCA data sets that include DD markers that incorrectly characterize the corresponding CA signals. One or more of the devices illustrated in FIG. 8 may further include a display configured to present information concerning at least one of the valid sub-set or invalid sub-set of the DCA data sets.

Additionally or alternatively, the one or more processors may be further configured to analyze a current one of the DCA data sets to extract one or more features of interest and to apply the one or more features of interest to the ML model. Additionally or alternatively, the one or more features of interest represent at least one of R-wave amplitude, R-wave amplitude variability, P-wave amplitude, P-wave amplitude variability, T-wave amplitudes, T-wave amplitude variability, RR interval amplitudes, RR interval amplitude variability, QRS area under the curve amplitudes, or QRS area under the curve amplitude variability, and the like. Additionally or alternatively, the CA signals represent subcutaneous electrocardiogram (EGM) signals for a series of beats over a predetermined period of time, the one or more processors configured to identify the one or more features of interest based in part on the CA signals aligned in time with the corresponding DD markers. Additionally or alternatively, the one or more devices of FIG. 8 implement an ML model that outputs, in connection with each DCA data set, at least one of: i) a confidence indicator indicative of a degree of confidence that the corresponding DCA data set represents a true positive or false positive designation of an arrhythmia of interest; ii) a confidence indicator indicative of an accuracy of R-wave sensing implemented by the IMD; iii) a recommendation indicative of a sensitivity level to be utilized by the IMD to identify R waves in the CA signals; or iv) an output indicating that a particular DCA data set is unduly noisy and should not be characterized as a normal sinus rhythm, nor an arrhythmia. One or more of the IMDs in FIG. 8 comprise a combination of subcutaneous electrodes configured to collect the CA signals; IMD memory configured to store program instructions; and one or more IMD processors configured to execute the program instructions to: analyze the CA signals and based on the analysis declare candidate arrhythmias episodes; generate the DCA data sets including the corresponding CA signals and the corresponding DD markers; and a transceiver configured to wirelessly transmit the DCA data sets to an external device. One or more of the external devices in FIG. 8 include the memory and the one or more processors and a transceiver, the transceiver configured to wirelessly receive the DCA data sets from the IMD. The server 802 may include memory and the one or more processors, the memory configured to store the collection of the DCA data sets, the one or more processors configured to apply the ML model to the collection of the DCA data sets.

The system of FIG. 8 further comprises one or more processors configured to execute the specific executable instructions to: obtain reference device classified arrhythmia (DCA) data sets associated with device declared arrhythmias, the reference DCA data sets including far field cardiac activity (CA) signals for one or more beats sensed by subcutaneous electrodes of an implantable medical device (IMD), the reference DCA data sets including one or more DD markers, generated by the IMD, characterizing the CA signals within the corresponding DCA data sets; generate synthetic DCA data sets based on the reference DCA data sets to form an augmented collection of DCA data sets; identify one or more features of interest from the augmented collection of DCA data sets; and apply the one or more features of interest to the ML model to train the ML model.

Additionally or alternatively, one or more processors of the system are further configured to apply a first augmented collection of the DCA data sets that represent valid DCA data sets that include DD markers that correctly characterize the corresponding CA signals; and to apply a second augmented collection of the DCA data sets that represent invalid DCA data sets that include DD markers that incorrectly characterize the corresponding CA signals. Additionally or alternatively, one or more processors of the system are further configured to generate the synthetic DCA data sets by at least one of shifting, rotating, stretching, shrinking or applying a Gaussian component to the reference DCA data sets. Additionally or alternatively, one or more processors of the system are further configured to generate the synthetic DCA data sets by shifting and wrapping the CA signals in the reference DCA data sets such that trailing portions of the reference DCA data sets are wrapped to form leading portions of the synthetic DCA data sets, and such that intermediate portions of the reference DCA data sets are shifted to form trailing portions of the synthetic DCA data sets. Additionally or alternatively, one or more processors of the system are further configured to generate the synthetic DCA data sets by at least one of stretching or shrinking the CA signals in the reference DCA data sets by at least one of adding or subtracting an amount of time to RR intervals between successive beats in the CA signals. Additionally or alternatively, the synthetic DCA data sets represent data sets that include artificially generated or computer-generated CA signals, where the CA signals and DD markers are based on the reference DCA data sets collected from a patient, but where the CA signals in the synthetic DCA data set are not collected from the patient.

FIG. 9 illustrates a system level diagram indicating potential devices and networks that utilize the methods and systems herein. For example, an IMD 902 may be utilized to collect a DCA data set. The IMD 902 may supply the DCA data set (CA signals, DD markers) as well as sensitivity levels and motion data, to various local external devices, such as a tablet device 904, a smart phone 906, a bedside monitoring device 908, a smart watch and the like. The devices 904-908 include a display to present the various types of the CA signals, DD markers, statistics (e.g., % valid, % invalid), diagnostics, recommendations for adjustments in IMD sensing/therapy parameters and other information described herein. The IMD 902 may convey the DCA data set over various types of wireless communications links to the devices 904, 906 and 908. The IMD 902 may utilize various communications protocols and be activated in various manners, such as through a Bluetooth, Bluetooth low energy, Wi-Fi or other wireless protocol. Additionally or alternatively, when a magnetic device 910 is held next to the patient, the magnetic field from the device 910 may activate the IMD 902 to transmit the DCA data set and arrythmia data to one or more of the devices 904-908.

In accordance with new and unique aspects herein, methods and systems are described to train and utilize machine learning models, such as a convolutional neural network (CNN), to determine whether candidate arrhythmias (e.g., AF), declared and classified by an IMD from subcutaneous EGMs (SEGMs), are true or false positives. Initial performance evaluation suggests that the CNN may reduce the false positive burden, related to ICM declared AF, by about 70% while still maintaining at least 98% sensitivity. The performance is similar to expert human adjudication. Further, the methods and systems herein are not limited to AF, but instead similar techniques can be applied to SEGMs for other arrhythmia types. Further, embodiments herein are not limited to CNN type machine learning models, but instead to be utilized with other machine learning models. For example, embodiments herein may train and utilize another machine learning model called a “gradient boosted decision tree” to classify asystole SEGMs. Embodiments herein combine both the firmware and machine learning discriminators into an optimal system for SEGM classification.

Embodiments may be implemented in connection with one or more implantable medical devices (IMDs). Non-limiting examples of IMDs include one or more of implantable leadless monitoring and/or therapy devices, and/or alternative implantable medical devices. For example, the IMD may represent a cardiac monitoring device, pacemaker, cardioverter, cardiac rhythm management device, defibrillator, leadless monitoring device, leadless pacemaker and the like. Additionally or alternatively, the IMD may be a leadless implantable medical device (LIMD) that include one or more structural and/or functional aspects of the device(s) described in U.S. Pat. No. 9,216,285 “LEADLESS IMPLANTABLE MEDICAL DEVICE HAVING REMOVABLE AND FIXED COMPONENTS” and U.S. Pat. No. 8,831,747 “LEADLESS NEUROSTIMULATION DEVICE AND METHOD INCLUDING THE SAME”, which are hereby incorporated by reference. Additionally or alternatively, the IMD may include one or more structural and/or functional aspects of the device(s) described in U.S. Pat. No. 8,391,980 “METHOD AND SYSTEM FOR IDENTIFYING A POTENTIAL LEAD FAILURE IN AN IMPLANTABLE MEDICAL DEVICE” and U.S. Pat. No. 9,232,485 “SYSTEM AND METHOD FOR SELECTIVELY COMMUNICATING WITH AN IMPLANTABLE MEDICAL DEVICE”, which are hereby incorporated by reference. Additionally or alternatively, the IMD may be a subcutaneous IMD that includes one or more structural and/or functional aspects of the device(s) described in U.S. application Ser. No. 15/973,195, titled “SUBCUTANEOUS IMPLANTATION MEDICAL DEVICE WITH MULTIPLE PARASTERNAL-ANTERIOR ELECTRODES” and filed May 7, 2018; U.S. application Ser. No. 15/973,219, titled “IMPLANTABLE MEDICAL SYSTEMS AND METHODS INCLUDING PULSE GENERATORS AND LEADS” filed May 7, 2018; U.S. application Ser. No. 15/973,249, titled “SINGLE SITE IMPLANTATION METHODS FOR MEDICAL DEVICES HAVING MULTIPLE LEADS”, filed May 7, 2018, which are hereby incorporated by reference in their entireties. Additionally or alternatively, the IMD may be a leadless cardiac monitor (ICM) that includes one or more structural and/or functional aspects of the device(s) described in U.S. Patent Application having Docket No. A15E1059, U.S. patent application Ser. No. 15/084,373, filed Mar. 29, 2016, entitled, “METHOD AND SYSTEM TO DISCRIMINATE RHYTHM PATTERNS IN CARDIAC ACTIVITY,”; U.S. patent application Ser. No. 15/973,126, titled “METHOD AND SYSTEM FOR SECOND PASS CONFIRMATION OF DETECTED CARDIAC ARRHYTHMIC PATTERNS”; U.S. patent application Ser. No. 15/973,351, titled “METHOD AND SYSTEM TO DETECT R-WAVES IN CARDIAC ARRHYTHMIC PATTERNS”; U.S. patent application Ser. No. 15/973,307, titled “METHOD AND SYSTEM TO DETECT POST VENTRICULAR CONTRACTIONS IN CARDIAC ARRHYTHMIC PATTERNS”; U.S. patent application Ser. No. 15/973,384, titled “METHOD AND SYSTEM TO DETECT NOISE IN CARDIAC ARRHYTHMIC PATTERNS”; and published as 2019/0336083A1 and U.S. patent application Ser. No. 16/930,791, filed Jul. 16, 2020, titled “METHODS, DEVICES AND SYSTEMS FOR HOLISTIC INTEGRATED HEALTHCARE PATIENT MANAGEMENT”, which are hereby incorporated by reference. Further, one or more combinations of IMDs may be utilized from the above incorporated patents and applications in accordance with embodiments herein.

All references, including publications, patent applications and patents, cited herein are hereby incorporated by reference to the same extent as if each reference were individually and specifically indicated to be incorporated by reference and were set forth in its entirety herein.

### Closing

The various methods as illustrated in the Figures and described herein represent exemplary embodiments of methods. The methods may be implemented in software, hardware, or a combination thereof. In various of the methods, the order of the steps may be changed, and various elements may be added, reordered, combined, omitted, modified, etc. Various of the steps may be performed automatically (e.g., without being directly prompted by user input) and/or programmatically (e.g., according to program instructions).

Various modifications and changes may be made as would be obvious to a person skilled in the art having the benefit of this disclosure. It is intended to embrace all such modifications and changes and, accordingly, the above description is to be regarded in an illustrative rather than a restrictive sense.

Various embodiments of the present disclosure utilize at least one network that would be familiar to those skilled in the art for supporting communications using any of a variety of commercially-available protocols, such as Transmission Control Protocol/Internet Protocol (“TCP/IP”), User Datagram Protocol (“UDP”), protocols operating in various layers of the Open System Interconnection (“OSI”) model, File Transfer Protocol (“FTP”), Universal Plug and Play (“UpnP”), Network File System (“NFS”), Common Internet File System (“CIFS”) and AppleTalk. The network can be, for example, a local area network, a wide-area network, a virtual private network, the Internet, an intranet, an extranet, a public switched telephone network, an infrared network, a wireless network, a satellite network and any combination thereof.

In embodiments utilizing a web server, the web server can run any of a variety of server or mid-tier applications, including Hypertext Transfer Protocol (“HTTP”) servers, FTP servers, Common Gateway Interface (“CGI”) servers, data servers, Java servers, Apache servers and business application servers. The server(s) also may be capable of executing programs or scripts in response to requests from user devices, such as by executing one or more web applications that may be implemented as one or more scripts or programs written in any programming language, such as Java®, C, C#or C++, or any scripting language, such as Ruby, PHP, Perl, Python or TCL, as well as combinations thereof. The server(s) may also include database servers, including without limitation those commercially available from Oracle®, Microsoft®, Sybase®, SAS® and IBM® as well as open-source servers such as MySQL, Postgres, SQLite, MongoDB, and any other server capable of storing, retrieving and accessing structured or unstructured data. Database servers may include table-based servers, document-based servers, unstructured servers, relational servers, non-relational servers or combinations of these and/or other database servers.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (“SAN”) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (“CPU” or “processor”), at least one input device (e.g., a mouse, keyboard, controller, touch screen or keypad) and at least one output device (e.g., a display device, printer or speaker). Such a system may also include one or more storage devices, such as disk drives, optical storage devices and solid-state storage devices such as random access memory (“RAM”) or read-only memory (“ROM”), as well as removable media devices, memory cards, flash cards, etc.

Such devices also can include a computer-readable storage media reader, a communications device (e.g., a modem, a network card (wireless or wired), an infrared communication device, etc.) and working memory as described above. The computer-readable storage media reader can be connected with, or configured to receive, a computer-readable storage medium, representing remote, local, fixed and/or removable storage devices as well as storage media for temporarily and/or more permanently containing, storing, transmitting and retrieving computer-readable information. The system and various devices also typically will include a number of software applications, modules, services or other elements located within at least one working memory device, including an operating system and application programs, such as a client application or web browser. It should be appreciated that alternate embodiments may have numerous variations from that described above. For example, customized hardware might also be used and/or particular elements might be implemented in hardware, software (including portable software, such as applets) or both. Further, connection to other computing devices such as network input/output devices may be employed.

Various embodiments may further include receiving, sending, or storing instructions and/or data implemented in accordance with the foregoing description upon a computer-readable medium. Storage media and computer readable media for containing code, or portions of code, can include any appropriate media known or used in the art, including storage media and communication media, such as, but not limited to, volatile and non-volatile, removable and non-removable media implemented in any method or technology for storage and/or transmission of information such as computer readable instructions, data structures, program modules or other data, including RAM, ROM, Electrically Erasable Programmable Read-Only Memory (“EEPROM”), flash memory or other memory technology, Compact Disc Read-Only Memory (“CD-ROM”), digital versatile disk (DVD) or other optical storage, magnetic cassettes, magnetic tape, magnetic disk storage or other magnetic storage devices or any other medium which can be used to store the desired information and which can be accessed by the system device. Based on the disclosure and teachings provided herein, a person of ordinary skill in the art will appreciate other ways and/or methods to implement the various embodiments.

The specification and drawings are, accordingly, to be regarded in an illustrative rather than a restrictive sense. It will, however, be evident that various modifications and changes may be made thereunto without departing from the broader spirit and scope of the invention as set forth in the claims.

Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions and equivalents falling within the spirit and scope of the invention, as defined in the appended claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including” and “containing” are to be construed as open-ended terms (i.e., meaning “including, but not limited to,”) unless otherwise noted. The term “connected,” when unmodified and referring to physical connections, is to be construed as partly or wholly contained within, attached to or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal.

Operations of processes described herein can be performed in any suitable order unless otherwise indicated herein or otherwise clearly contradicted by context. Processes described herein (or variations and/or combinations thereof) may be performed under the control of one or more computer systems configured with executable instructions and may be implemented as code (e.g., executable instructions, one or more computer programs or one or more applications) executing collectively on one or more processors, by hardware or combinations thereof. The code may be stored on a computer-readable storage medium, for example, in the form of a computer program comprising a plurality of instructions executable by one or more processors. The computer-readable storage medium may be non-transitory.

All references, including publications, patent applications and patents, cited herein are hereby incorporated by reference to the same extent as if each reference were individually and specifically indicated to be incorporated by reference and were set forth in its entirety herein.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and physical characteristics described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112 (f), unless and until such claim limitations expressly use the phrase “means for” followed by a statement of function void of further structure.

What is claimed is:

1. A system, comprising:
memory configured to store specific executable instructions; and
one or more processors configured to execute the specific executable instructions to:
obtain reference cardiac activity (CA) signals for one or more beats sensed by electrodes of a medical device;
generate an augmented collection of data sets based on the reference CA signals;
apply the augmented collection of data sets to a machine learning (ML) model to train the ML model; and
apply new CA signals to the ML model to obtain an indication of whether the new CA signals correspond to an arrhythmia or normal sinus rhythm.

2. The system of claim 1, wherein the one or more processors are further configured to generate the augmented collection of data sets by at least one of shifting, rotating, stretching, shrinking or applying a noise component to the reference CA signals in order to enlarge a total number of CA signals, within the augmented collection of data sets, beyond the reference CA signals thereby avoid overfitting while training the ML model.

3. The system of claim 1, wherein the one or more processors are further configured to generate the augmented collection of data sets by applying noise to the reference CA signals in order to enlarge a total number of CA signals, within the augmented collection of data sets, beyond the reference CA signals thereby avoid overfitting while training the ML model.

4. The system of claim 1, wherein the one or more processors are further configured to obtain reference device classified arrhythmia (DCA) data sets associated with device declared arrhythmias, the reference DCA data sets including the reference CA signals for one or more beats sensed by subcutaneous electrodes of an implantable medical device (IMD), the reference DCA data sets including one or more device documented (DD) markers, generated by the IMD, characterizing the reference CA signals within the corresponding DCA data sets.

5. The system of claim 4, wherein the one or more processors are further configured to apply a first augmented collection of data sets that represent valid DCA data sets that include DD markers that correctly characterize the corresponding CA signals; and to apply a second augmented collection of data sets that represent invalid DCA data sets that include DD markers that incorrectly characterize the corresponding CA signals.

6. The system of claim 1, wherein the one or more processors are further configured to generate the augmented collection of data sets by shifting and wrapping the reference CA signals such that trailing portions of the reference CA signals are wrapped to form leading portions of the augmented collection of data sets, and such that intermediate portions of the reference CA signals are shifted to form trailing portions of the augmented collection of data sets in order to enlarge a total number of CA signals, within the augmented collection of data sets, beyond the reference CA signals thereby avoid overfitting while training the ML model.

7. The system of claim 1, wherein the one or more processors are further configured to generate the augmented collection of data sets by at least one of stretching or shrinking the reference CA signals by at least one of adding or subtracting an amount of time to RR intervals between successive beats in the reference CA signals in order to enlarge a total number of CA signals, within the augmented collection of data sets, beyond the reference CA signals thereby avoid overfitting while training the ML model.

8. The system of claim 1, wherein the augmented collection of data sets represent data sets that include artificially generated or computer-generated CA signals that are based on the reference CA signals collected from a patient in order to enlarge a total number of CA signals, within the augmented collection of data sets, beyond the reference CA signals thereby avoid overfitting while training the ML model.

9. The system of claim 1, wherein the medical device is implantable, the one or more processors are further configured to adjust at least one of sensing or therapy parameters of the implantable medical device based on the indication.

10. The system of claim 1, wherein the medical device is implantable, the one or more processors are further configured to deliver a therapy based on the indication.

11. A computer implemented method for building a machine learning (ML) model to confirm device documented (DD) arrhythmias, comprising:
under control of one or more processors configured with specific executable instructions,
obtaining reference cardiac activity (CA) signals for one or more beats sensed by electrodes of a medical device;
generating an augmented collection of data sets based on the reference CA signals;
applying the augmented collection of data sets to a machine learning (ML) model to train the ML model; and
applying new CA signals to the ML model to obtain an indication of whether the new CA signals correspond to an arrhythmia or normal sinus rhythm.

12. The method of claim 11, wherein the generating the augmented collection of data sets includes at least one of shifting, rotating, stretching, shrinking or applying a noise component to the reference CA signals in order to enlarge a total number of CA signals, within the augmented collection of data sets, beyond the reference CA signals thereby avoid overfitting while training the ML model.

13. The method of claim 11, wherein the generating the augmented collection of data sets includes applying noise to the reference CA signals in order to enlarge a total number of CA signals, within the augmented collection of data sets, beyond the reference CA signals thereby avoid overfitting while training the ML model.

14. The method of claim 11, further comprising obtaining reference device classified arrhythmia (DCA) data sets associated with device declared arrhythmias, the reference DCA data sets including the reference CA signals for one or more beats sensed by subcutaneous electrodes of an implantable medical device (IMD), the reference DCA data sets including one or more device documented (DD) markers, generated by the IMD, characterizing the reference CA signals within the corresponding DCA data sets.

15. The method of claim 14, further comprising applying a first augmented collection of data sets that represent valid DCA data sets that include DD markers that correctly characterize the corresponding CA signals; and applying a second augmented collection of data sets that represent invalid DCA data sets that include DD markers that incorrectly characterize the corresponding CA signals.

16. The method of claim 11, wherein the generating the augmented collection of data sets includes shifting and wrapping the reference CA signals such that trailing portions of the reference CA signals are wrapped to form leading portions of the augmented collection of data sets, and such that intermediate portions of the reference CA signals are shifted to form trailing portions of the augmented collection of data sets in order to enlarge a total number of CA signals, within the augmented collection of data sets, beyond the reference CA signals thereby avoid overfitting while training the ML model.

17. The method of claim 11, wherein the generating the augmented collection of data sets includes at least one of stretching or shrinking the reference CA signals by at least one of adding or subtracting an amount of time to RR intervals between successive beats in the reference CA signals in order to enlarge a total number of CA signals, within the augmented collection of data sets, beyond the reference CA signals thereby avoid overfitting while training the ML model.

18. The method of claim 11, wherein the augmented collection of data sets represents data sets that include artificially generated or computer-generated CA signals that are based on the reference CA signals collected from a patient in order to enlarge a total number of CA signals, within the augmented collection of data sets, beyond the reference CA signals thereby avoid overfitting while training the ML model.

19. The method of claim 11, wherein the medical device is implantable, the method further comprising adjusting at least one of sensing or therapy parameters of the implantable medical device based on the indication.

20. The method of claim 11, wherein the medical device is implantable, the method further comprising delivering a therapy by the implantable medical device based on the indication.
